(19) United States
(12) Patent Application Publication
(10) Pub. No.: US 2008/0261821 A1
(43) Pub. Date: Oct. 23, 2008

(54) Mechanism-Based Crosslinkers

(75) Inventors: Dustin Maly, San Francisco, CA (US); Kevan M. Shokat, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: THE REGENTS OF TE UNIVERSITY OF CALIFORNIA, Oakland, CA (US)

(21) Appl. No.: 11/572,082

(22) PCT Filed: Jul. 21, 2005
(86) PCT No.: PCT/US2005/026359

§ 371 Date: Jun. 20, 2008

Related U.S. Application Data
(60) Provisional application No. 60/590,304, filed on Jul. 21, 2004.

(51) Int. Cl. C40B 30/04; G01N 33/573

(52) U.S. Cl. 506/9; 435/7.4; 536/27.3

(57) ABSTRACT

The present invention provides novel mechanism-based crosslinkers useful in covalently linking a kinase and an interactor.

### CROSS-REFERENCES TO RELATED APPLICATIONS

This application claims the benefit of U.S. Provisional Patent Application No. 60/590,304, filed Jul. 21, 2004, which is incorporated herein by reference in its entirety for all purposes.

### STATEMENT AS TO RIGHTS TO INVENTIONS MADE UNDER FEDERALLY SPONSORED RESEARCH AND DEVELOPMENT

The present invention was supported by a grant from the National Institutes of Health (RO1EB001987). The Government may have certain rights to the invention.

### BACKGROUND OF THE INVENTION

Protein phosphorylation is the dominant form of information transfer in cells and the dissection of phosphorylation cascades is essential for our understanding of signal transduction (Manning et al., Science, 298, 1912 (2002)). The protein kinases are one of the largest protein super-families in eukaryotic genomes and have attracted considerable scientific interest because of their roles in cell physiology and pathophysiology (Hunter, T., Cell, 100, 113-127 (2000)). There are approximately 600 protein kinases and thousands of phosphorylated proteins (many on multiple sites) in humans. For most kinases the true protein targets are unknown.

One challenge in the area of protein kinase inhibitor discovery is the development of new classes of kinase inhibitors. The vast majority of known kinase inhibitors bind in the highly conserved ATP binding pocket of kinases, resulting in less than perfectly specific inhibitors of individual protein kinases. Inhibitors which bind outside of the pocket occupied by ATP-proper, such as Gleevec have demonstrated the potential advantages of this approach. The binding pocket, or groove, (also referred to herein as the peptide binding groove) for the second substrate, the protein, has been largely unexplored in this regard. The difficulty is two-fold: 1) most drug libraries contain heterocyclic compounds that exhibit ideal binding to the adenine binding pocket and not the peptide/protein surface of the second substrate; and 2) the peptide binding groove is a challenge to traditional enzyme inhibitor discovery approaches as evidenced by the challenge of inhibiting protein-protein interactions. One way to search for small molecules which bind to the peptide/protein binding substrate pocket of kinases (e.g. interactors) is to use a covalent bond to direct potential inhibitors to the desired site. This approach has two advantages in that the first inhibitors may be weak (e.g. IC50 in the μM or mM range) and thus the use of a covalent bond to trap weak binders may provide the early SAR necessary to optimize early hit compounds. Secondly, the covalent bond forming strategy serves as a directing group to position candidate molecules into the peptide binding groove rather than the ATP binding pocket.

Despite the development of a variety of creative and powerful new technologies to understand their function (Shogren-Knaak et al., Annu. Rev. Cell Dev. Biol., 17, 405-433 (2001); Williams, D. M. and Cole, P. A., Trends Biol. Sci., 26, 271-273 (2001); Parang et al., FEBS Lett., 520, 156-160 (2002)), key cellular protein targets remain elusive. Unlike many protein-protein interactions such as those underlying SH2 domain-phosphotyrosine and 14-3-3-phosphoserine recognition (Fu et al., Annu. Rev. Pharmacol. Toxicol., 40, 617-647 (2000); Kuriyan, J. and Cowburn, D., Annu. Rev. Biophys. Biomol. Struct., 26, 259-288 (1997)), protein kinase-protein substrate interactions are often low affinity and not reliably discovered by techniques such as affinity pull-downs or two-hybrid screens.

Biochemical and proteomic efforts have been invaluable in identifying numerous phosphoproteins and phosphorylation sites (McLachlin et al., Curr. Opin. Chem. Biol. 5, 591-602 (2001), Mann et al. Trends Biotechnol., 20, 261-268 (2002), Knight et al., Nat. Biotechnol. 21, 1047-10 (2003)). Unfortunately, the full potential of this information remains unrealized because there is a dearth of techniques that allow a researcher to use a phosphoprotein to identify its upstream kinase (Parang et al., FEBS Lett. 520, 156-160 (2002), Shen et al., Chem. Soc. 125, 16172-16173 (2003)).

By providing mechanism-based crosslinkers capable of covalently linking kinases and interactors, the present invention fulfills these and other needs in the art.

### BRIEF SUMMARY OF THE INVENTION

The present invention provides novel mechanism-based crosslinkers capable of covalently linking a kinase with an interactor. The mechanism-based crosslinkers of the present invention provide a completely new modality in enzyme crosslinking.

In some embodiments, the mechanism-based crosslinker of the present invention has the formula:

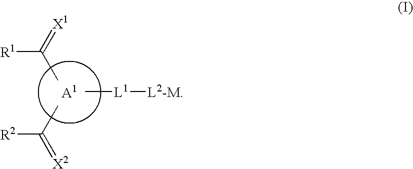

In Formula (I), X¹ and X² are independently O, S, or N. M is an ATP-binding moiety.

R¹ and R² are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A¹ is substituted or unsubstituted cycloalkyl, substituted or unsubstituted heterocycloalkyl, substituted or unsubstituted aryl, substituted or unsubstituted heteroaryl, or substituted or unsubstituted fused ring.

L² is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L¹ is a bond, —C(O)-L³-, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L³ is a bond, —O-L⁴-, or —N(R⁵)-L⁴-. R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L⁴ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In some embodiments, the mechanism-based crosslinker has the formula:

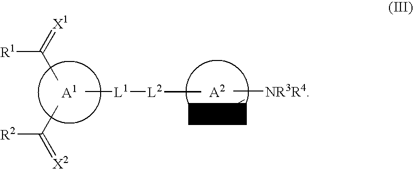

In Formula (III), R¹, R², A¹, X¹, X², L¹, and L² are as described above in the discussion of Formula (I).

R³ and R⁴ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, the mechanism-based crosslinker has the formula:

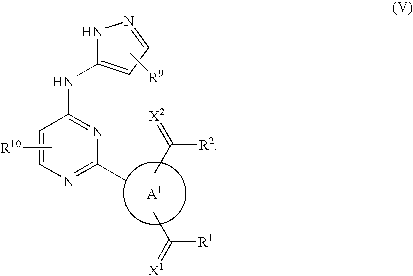

In Formula (V), X¹, X², R¹, R², and A¹ are as defined in the discussion of Formula (I).

R⁹ and R¹⁰ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In another aspect, the present invention provides a method of detecting binding between an interactor and a kinase. The method includes contacting a kinase with a mechanism-based crosslinker and an interactor. The mechanism-based crosslinker is allowed to form a covalent bond with the interactor. The mechanism-based crosslinker is also allowed to specifically form a covalent bond with a catalytic amino acid side chain of the kinase thereby forming a crosslinked kinase-interactor pair. The presence of the crosslinked kinase-interactor pair is then detected, thereby detecting the binding between the interactor and the kinase.

In another aspect, the present invention provides a method of detecting an active kinase in a sample. The method includes contacting an array of immobilized interactors with a mechanism-based crosslinker and a sample comprising an active kinase. The method also includes allowing the mechanism-based crosslinker to form a covalent bond with the interactor and specifically form a covalent bond with a catalytic amino acid side chain of the active kinase. An immobilized crosslinked kinase-interactor pair is thereby formed. Finally, the presence of the immobilized crosslinked kinase-interactor pair is detected thereby detecting said active kinase.

### BRIEF DESCRIPTION OF THE DRAWINGS

FIG. 1 illustrates a schematic representation of a crosslinking reaction.

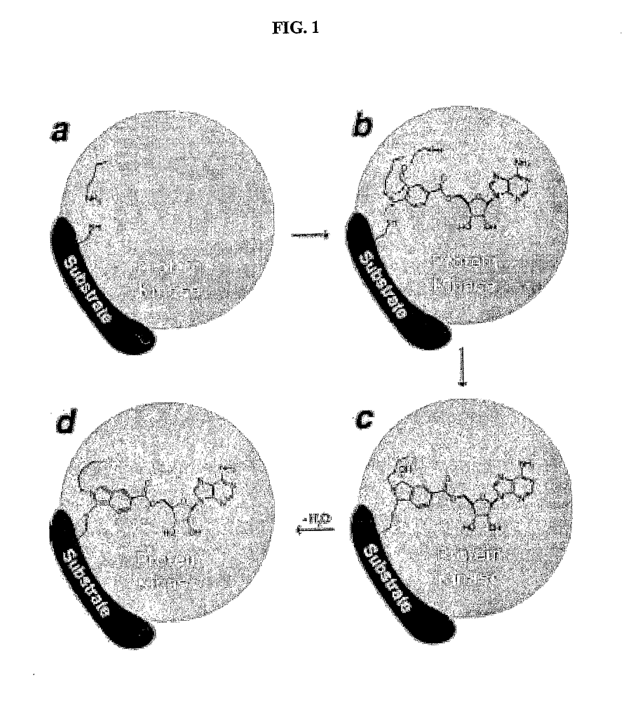

FIG. 2 illustrates an SDS-PAGE analysis of a crosslinking reaction showing the (A) initial characterization of the proposed crosslinking reaction, (B) crosslinking with peptide derivatives, and (C) crosslinking with aldehyde derivatives.

FIG. 3 illustrates the kinetics of the crosslinking reaction with 1 μM fluorescein-ZZRPRTSCF-OH (6), AKT1 (60 nM), dialdehyde 2 (20 μM), and BME (20 μM) for 5-80 min at room temperature (separation by SDS-PAGE).

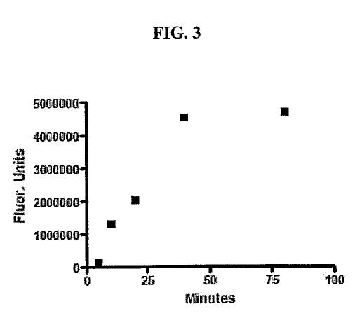

FIG. 4 illustrates an electrophoretic gel showing a peptide-AKT1 complex running at a slightly higher molecular weight than AKT1 after incubation of AKT1 (150 nM) and peptide 5 (1 μM) for 20 min at room temperature in the presence (lane 2) or absence (lane 1) of dialdehyde 2 (20 μM) (the crude reactions were resolved by SDS-PAGE and the gel was stained with Sypro Ruby protein stain).

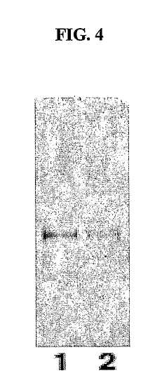

FIG. 5 illustrates the effect of exogenous thiols on crosslinking efficiency wherein 1 μM fluorescein-ZZRPRTSCF-OH (6) was incubated with AKT1 (60 nM), dialdehyde 2 (100 μM), and BME (Left) or Ac-Cys-OH (0-100 μM) (right) for 20 min at room temperature resulting from SDS-PAGE analysis of the crosslinking reactions followed by fluorescence imaging (excitation at 480 nM, emission collected with a 520 nM band pass filter) (FU=fluorescence units, Fl-AKTide-SH=fluorescein-ZZRPRTSCF-OH (6)) performed in triplicate.

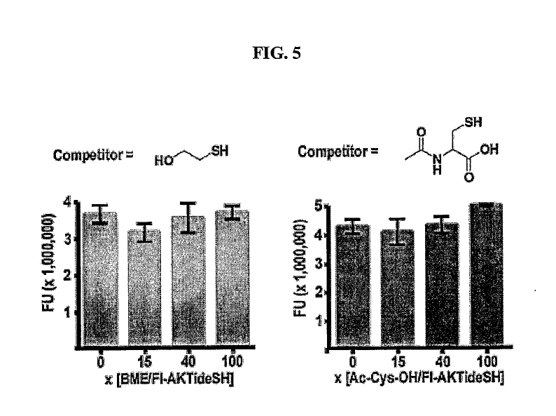

FIG. 6 illustrates the effect of ATP and Biotin-ZRPRTSSF-OH, wherein (A) 5 μM fluorescein-ZZRPRTSCF-OH (6) was incubated with AKT1 (60 mM), dialdehyde 2 (100 μM), and serine peptide 3 (0.0004-5 mM) for 20 min at room temperature, and (B) 5 μM peptide 6 was incubated with AKT1 (60 nM), dialdehyde 2 (20 μM), and ATP (0.01-20 mM) for 20 min at room temperature, resulting from SDS-PAGE analysis of the crosslinking reactions followed by fluorescence imaging (excitation at 480 nM, emission collected with a 520 nM band pass filter)(% Max. Fluor.=the number of fluorescence units measured per concentration of competitor divided by the number of fluorescence units measured with no competitor multiplied by 100) performed in triplicate.

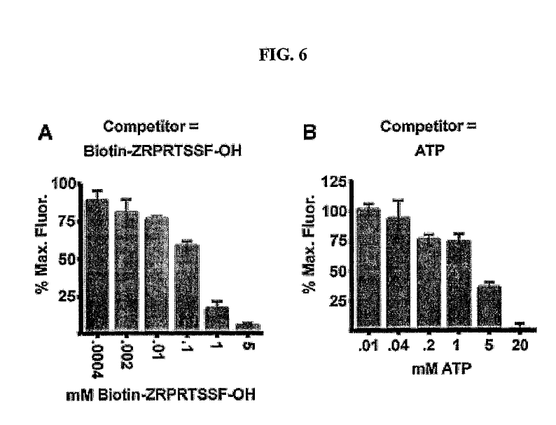

FIG. 7 illustrates SDS-PAGE analysis of the crosslinking reaction for other serine/threonine kinases after gels were transferred to nitrocellulose and probed for biotin with streptavidin-HRP, where (A) 5 μM Biotin-ZLRRAXLG-OH (X═C or S) was incubated with PKA (175 nM) and dialdehyde 2 (20 μM) for 20 min at room temperature, (B) 20 μM Biotin-ZIPTXPITTTYF-OH(X═C or S) was incubated with p38-as1 (500 nM) and dialdehyde 2 (100 μM) for 60 min at room temperature, or (C) 20 μM of Biotin-ZRRADDXDDDD-OH (X═C or S) was incubated with Casein Kinase II (150 nM) and dialdehyde 2 (100 μM) for 60 min at room temperature.

### DETAILED DESCRIPTION OF THE INVENTION

### Abbreviations and Definitions

The abbreviations used herein have their conventional meaning within the chemical and biological arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e. unbranched) or branched chain, or cyclic hydrocarbon radical, or combination thereof, which may be fully saturated, mono- or polyunsaturated and can include di- and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—, and further includes those groups described below as “heteroalkylene.” Typically, an alkyl (or alkylene) group will have from 1 to 24 carbon atoms, with those groups having 10 or fewer carbon atoms being preferred in the present invention. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen and sulfur atoms may optionally be oxidized and the nitrogen heteroatom may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂-O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂-S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent radical derivatives of “cycloalkyl” and “heterocycloalkyl,” respectively.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (preferably from 1 to 3 rings) which are fused together or linked covalently. The term “heteroaryl” refers to aryl groups (or rings) that contain from one to four heteroatoms selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. The terms “arylene” and “heteroarylene” refer to the divalent radical derivatives of “aryl” and “heteroaryl,” respectively.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

The term “oxo” as used herein means an oxygen that is double bonded to a carbon atom.

A “fused ring” refers to multiple rings (e.g. 1 to 3 rings) which are fused (i.e. linked covalently at two or more adjacent ring vertices). The fused rings are fused substituted or unsubstituted cycloalkyls, substituted or unsubstituted heterocycloalkyls, substituted or unsubstituted aryls, substituted or unsubstituted heteroaryls, and/or combinations thereof.

Each of the above terms (e.g., “alkyl,” “heteroalkyl,” “aryl” and “heteroaryl”) are meant to include both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)NR″″, —NR—C(NR′R″)=NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: halogen, —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)=NR′R″, —NR—C(NR′R″)=NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present.

Two of the substituents on adjacent atoms of the aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B-, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

A “substituent group,” as used herein, means a group selected from the following moieties:
(A) —OH, —N—H₂, —SH, —CN, —CF₃, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxy, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from oxo, —OH, —NH₂, —SH, —CN, —CF₃, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The compounds of the present invention may exist as salts. The present invention includes such salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, CF₃CO₂H (e.g. 2CF₃CO₂H·H₂O) and the like. Also included are salts of amino acids such as arginate and the like, and salts of organic acids like glucuronic or galactunoric acids and the like. Certain specific compounds of the present invention contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, tautomers, geometric isomers and individual isomers are encompassed within the scope of the present invention. The compounds of the present invention do not include those which are known in the art to be too unstable to synthesize and/or isolate.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are encompassed within the scope of the present invention.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

The term “mechanism-based crosslinker,” as used herein, means a compound capable of covalently linking a kinase and an interactor by forming a covalent bond with the interactor and specifically forming a covalent bond with a catalytic amino acid side chain of the kinase.

The term “specifically form a covalent bond with a catalytic amino acid side chain of a kinase” as used herein, means that a covalent bond is preferentially formed to a catalytic amino acid side chain of a kinase as compared to a non-catalytic amino acid side chain of a kinase. A “catalytic amino acid side chain of a kinase” is an amino acid side chain in the active site of a kinase that participates in the catalytic mechanism of phosphoryl transfer from a nucleotide triphosphate to a metabolite. In some embodiments, the catalytic amino acid side chain of a kinase is a lysine side chain of a kinase.

“Peptide” refers to a polymer in which the monomers are amino acids and are joined together through amide bonds, alternatively referred to as a “polypeptide.” The terms “peptide” and “polypeptide” encompass proteins. Unnatural amino acids, for example, β-alanine, phenylglycine and homoarginine are also included under this definition. Amino acids that are not gene-encoded may also be used in the present invention. Furthermore, amino acids that have been modified to include reactive groups may also be used in the invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

The term “interactor” as used herein refers to a compound capable of binding to a kinase. Typically, interactors bind to the peptide binding groove of the kinase. An interactor may be a kinase inhibitor or kinase substrate. In some embodiments, interactors are peptides.

An “amino” as used herein, is a monovalent radical having the formula —NR′R″. R′ and R″ may independently by hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “sulfhydryl,” as used herein, is a monovalent radical having the formula —SH.

A “kinase,” as used herein, is an enzyme that is capable of catalyzing the transfer of a phosphoryl group (also referred to as a phosphate group) from a nucleoside triphosphate to another compound. Typically, the kinase transfers the phosphoryl group (e.g. a terminal phosphate group) from adenosine triphosphate (ATP) to another compound. An active kinase is a kinase with detectable catalytic activity.

The term “luminescent” as used herein, refers to the ability of a compound or complex to emit light in response to energy (such as electrical, chemical or light energy) other than thermal energy. For example, a luminescent complex includes fluorescent and phosphorescent complexes or compounds but not incandescent complexes or compounds. Thus, “luminescence,” as used herein, refers to the emission of light from a luminescent compound upon contact with energy, such as electrical, chemical or light energy, other than thermal energy.

The step of “contacting” a kinase, interactor, or mechanism-based crosslinker to form a covalent bond with a recited species is conducted under reaction conditions suitable for covalent bond formation. One skilled in the art will easily recognize or determine the appropriate conditions. In some embodiments, the reaction conditions include an aqueous environment.

As used herein, the symbol [symbol] denotes the point of attachment of a chemical moiety to the remainder of the molecule.

### Introduction

The present invention provides compositions and methods of using a novel mechanism-based crosslinker capable of covalently linking a kinase with an interactor. Unlike previously known enzyme crosslinkers (e.g. Parang et al., FEBS Lett. 520: 156-160 (2002)), the mechanism-based crosslinkers described herein are selectively activated by the kinase catalytic machinery. By utilizing the catalytic machinery, these mechanism-based crosslinkers provide increased selectivity by requiring proper orientation of the interactor in relation to the kinase active site before crosslinking may occur. Moreover, the mechanism-based crosslinkers may be used to distinguish between inactive kinases and active kinases.

Thus, the mechanism-based crosslinkers of the present invention provide a completely new modality in enzyme crosslinking. The mechanism-based crosslinkers uniquely provide specificity in forming covalent bonds at defined kinase catalytic amino acids and kinase interactor phosphorylation sites while simultaneously allowing generalized crosslinking between kinases and interactors by exploiting known kinase catalytic paradigms.

### I. Mechanism-Based Crosslinkers

Mechanism-based crosslinkers of the present invention are capable of forming a covalent bond between an interactor and a catalytic amino acid side chain of a kinase. Typically, the mechanism-based cross-linker will specifically form the covalent bond with the catalytic amino acid side chain of a kinase. In some embodiments, the covalent bond with the catalytic amino acid is formed only in the presence of the interactor.

The mechanism-based crosslinkers described herein may contain reactive moieties (also referred to herein as crosslinker reactive groups) that form covalent bonds with the catalytic amino acid side chain of a kinase and/or the interactor. Any appropriate number of crosslinker reactive groups may be present. In some embodiments, the mechanism-based crosslinker includes a single crosslinker reactive group that reacts with both the catalytic amino acid side chain of a kinase and the interactor. In other embodiments, the mechanism-based crosslinker includes two or more crosslinker reactive groups. In a related embodiment, at least one of the crosslinker reactive groups forms a covalent bond with the interactor and specifically forms a covalent bond with the catalytic amino acid side chain of a kinase. Alternatively, at least one first crosslinker reactive group forms a covalent bond with the interactor and at least one second crosslinker reactive group specifically forms a covalent bond with the catalytic amino acid side chain of a kinase. Other crosslinker reactive groups may form covalent bonds with other catalytic or non-catalytic amino acid side chains of the kinase and/or the interactor.

The mechanism-based crosslinkers may form a covalent bond with any appropriate catalytic amino acid side chain of a kinase and any appropriate chemical moiety of the interactor. The chemical moiety of the interactor that forms a covalent link with the crosslinker may also be referred to herein as an interactor reactive group. The chemical moiety of the amino acid side chain of a kinase that forms a covalent link with the crosslinker may also be referred to herein as a side chain reactive group. Typically, the amino acid side chain and/or interactor reactive groups are nucleophilic. Nucleophilic reactive groups are well known in the art and include, for example, sulfhydryls, aminos, hydroxyls (e.g. alcohols), carboxyls, and salts thereof.

In some embodiments, the interactor reactive group and the side chain reactive group are independently an amino or sulfhydryl. In some embodiments, the interactor reactive group is a sulfhydryl and the side chain reactive group is an amino. In other embodiments, the interactor group is a sulfhydryl and the catalytic amino acid side chain is a lysine side chain.

In some embodiments, the mechanism-based crosslinker includes a first crosslinker reactive group and a second crosslinker reactive group. In a related embodiment, the first crosslinker reactive group and the second crosslinker reactive group are an aldehyde. In another related embodiment, the first crosslinker reactive group forms a covalent bond with the catalytic amino acid side chain and the second crosslinker reactive group.

The mechanism-based crosslinkers of the present invention may include an ATP-binding moiety. An ATP-binding moiety is capable of binding to the ATP-binding pocket of a kinase. Binding of the ATP-binding moiety to the ATP-binding pocket of a kinase may be accomplished using any appropriate binding interaction, such as hydrogen bonding, Van der Waals forces, hydrophobic interactions, pi-pi interactions, ionic bonding, dipole-dipole interactions, or combinations thereof.

Kinase ATP-binding pockets and ATP-binding moieties are well known in the art and are described in detail below. In some embodiments, the ATP-binding moiety is an adeninyl moiety, an adenosinyl moiety, or a 2′-deoxy-adenosinyl moiety, or derivative thereof.

Thus, in some embodiments, the mechanism-based crosslinkers of the present invention include a first crosslinker reactive group and a second crosslinker reactive group that are both aldehyde groups. The first aldehyde crosslinker reactive group is capable of forming a covalent bond with both the catalytic amino acid side chain and the interactor. In this embodiment, the catalytic amino acid side chain includes an amino or sulfhydryl side chain reactive group. The interactor includes an amino or sulfhydryl interactor reactive group. In a related embodiment, the interactor reactive group is a sulfhydryl and the amino acid side chain group is an amino. In another related embodiment, the mechanism-based crosslinker includes an ATP-binding moiety. The ATP-binding moiety may be an adeninyl moiety, adenosinyl moiety, 2′-deoxy-adenosinyl moiety, or derivative thereof generally known in the art.

ATP-Binding Moieties

As described above, ATP-binding moieties are those chemical moieties generally known in the art that are capable of binding to the ATP-binding pocket of a kinase using any appropriate intermolecular binding interaction, such as hydrogen bonding, Van der Waals forces, hydrophobic interactions, pi-pi interactions, ionic bonding, dipole-dipole interactions, or combinations thereof.

A wide variety of ATP-binding moieties are useful in the present invention. Typically, ATP-binding moieties mimic mainly the adenine portion of ATP. Adenine has been described as a fuzzy recognition template (Moodie et al., J. Mol. Biol., 263, 486 (1996)). ATP binds to kinases in a uniform manner in a cleft between the two kinase lobes. In nearly all known cases, a tridentate H-bonding motif facilitates the interaction between the ATP-binding moiety and the ATP-binding pocket. The H-bonding motif includes the backbone amide bonds of the hinge region, which is a short segment connecting the N- and C-terminal kinase lobes. The H-bonding motif also includes the purine N⁶H₂ (donor), N¹ (acceptor), and C²H (donor) groups. The donor-acceptor-donor H-bonding motif of the ATP aminopyrimidine ring is usually mirrored in heterocyclic inhibitors in a variety of guises (Wu et al., Structure, 11, 399 (2003)). The remaining electrostatic interactions between kinases and ATP may involve the ribose and triphosphate moieties.

Extending in the plane of the purine ring system, a hydrophobic pocket and a hydrophobic channel leading to the solvent-exposed entrance to the ATP-binding pocket is generally present in the ATP-binding pocket. Although neither of these sites are occupied in a significant manner by ATP, they may be occupied ATP-binding moieties. Although subtle variations in the overall disposition of van der Waals and lipophilic elements may exist, ATP-binding pocket is essentially invariant (Engh, R. A. and Bossemeyer, D., Pharmacol. Ther., 93, 99 (2002)).

Thus, ATP-binding moieties useful in the present invention typically include those moieties capable of participating in the tridentate H-bonding within the ATP-binding pocket. The ATP-binding moieties may include a hydrogen donor at the appropriate position to electronically mimic the purine N⁶H₂ (donor). The ATP-binding moiety may also include at the appropriate position a hydrogen acceptor to electronically mimic the purine N¹. In some embodiments, the ATP-binding moiety includes a hydrogen donor at the appropriate position to electronically mimic the purine C²H group.

Useful ATP-binding moieties include adeninyl moieties, adenosinyl moieties, 2′-deoxy-adenosinyl moieties, and known derivatives thereof. Exemplary ATP-binding moieties useful in the present invention are summarized in detail in Fischer, Current Medicinal Chemistry, 11, 1563-1583 (2004), and include, ATP-binding moieties that form at least a portion of the following inhibitors: the anilinopyrimidine compound STI-571 (imatinib) (Fabbro et al., Pharmacol. Ther., 93, 79 (2002)) (see also below); pyridinylimidazole inhibitors (Wilson et al., Chem. Biol., 4, 423 (1997); Wang et al., Structure, 6, 1117 (1998)); ATP-uncompetitive inhibitors, such as PD 184352, now under clinical investigation as CI-1040 (Sebolt-Leopold, J. S., Oncogene, 19, 6594 (2000)), Sebolt-Leopold, J. S. et al., Nat. Med., 5, 810 (1999)); Analogue-sensitive kinase alleles (ASKA) and their selective inhibitors (Bishop et al., Nature, 407, 395 (2000), Witucki et al., Chem. Biol., 9, 25 (2002)), CDK inhibitors (Fischer, P. M. and Gianella-Borradori, A., Exp. Opin. Investig. Drugs, 12, 955 (2003), Fischer et al., In Progress in Cell Cycle Research, Meijer, L. et al., eds. (Vol. 5) pp. 235-248, Editions de la Station Biologique de Roscoff (2003), 2-anilino-4-triazolopyrimidines (Wu et al., Structure, 11, 399 (2003)), the aloisines (Mettey et al., J. Med. Chem., 46, 222 (2003)), certain 1H-pyrazolo[3,4-b]pyridines (Misra et al., Bioorg. Med. Chem. Lett., 13, 1133 (2003)), and oxindoles (Dermatakis et al., Bioorg. Med. Chem., 11, 1873 (2003))); GSK3/CDK inhibitors (e.g. paullones (Leost et al., Eur. J. Biochem., 267, 5983 (2000), Knockaert et al., J. Biol. Chem., 277, 25493 (2002)), hymenialdisine (Meijer et al., Chem. Biol., 7, 51 (2000)), indirubins (Damiens et al., Oncogene, 20, 3786 (2001)), and aloisines (Mettey et al., J. Med. Chem., 46, 222 (2003)); triarylpyrimidines, e.g. CHIR-98023 (Nikoulina et al., Diabetes, 51, 2190 (2002); Cline et al., Diabetes, 51, 2903 (2002); Henriksen et al., Am. J. Physiol, 284, E892 (2003)), 5-aryl-pyrazolo[3,4-b]pyridines (Witherington et al., Bioorg. Med. Chem. Lett., 13, 1577 (2003)); thiadiazolidinones (Martinez et al., J. Med. Chem., 45, 1292 (2002)); scytonemin (Stevenson et al., J. Pharmacol. Exp. Ther., 303, 858 (2002); Stevenson et al., Inflamm. Res., 51, 112 (2002)); staurosporine and the dimethoxyquinazoline (Engh et al., J. Biol. Chem., 271, 26157 (1996); Xu et al., Proc. Natl. Acad. Sci. USA, 93, 6308 (1996)); quinazoline derivative inhibitors of both aurora-A and B kinases (Ditchfield et al., J. Cell. Biol., 161, 281 (2003), Bridges, A. J., Chem. Rev., 101, 2541 (2001)); Hesperadin (Hauf et al., J. Cell. Biol., 161, 281 (2003)); inhibitors of the c-Src PTK (Maly et al., Proc. Natl. Acad. Sci. USA, 97, 2419 (2000)); O-methyl oxime monomer fragments; and adenosine kinase (AK) inhibitors such as pyridopyrimidine (Williams et al., J. Med. Chem., 42, 1481 (1999)).

Other useful ATP-binding moieties are discussed in detail in WO 02/062789, which is herein incorporated by reference in its entirety for all purposes.

Based on the known correlation between the structures of ATP-binding moieties and the ability of those structures to bind to the known structures of ATP-binding pockets, one skilled in the art will readily recognize and visualize the ATP-binding moieties of the present invention.

In some embodiments, the ATP-binding moiety, or a portion thereof, has the formula:

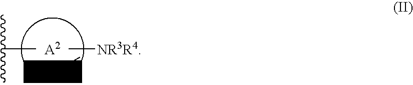

In Formula (II), A², R³, and R⁴ are as defined in the discussion of the compounds of Formula (I) below. In some embodiments, at least one of R³ or R⁴ is a hydrogen capable of participating in the tridentate H-bonding within the ATP-binding pocket.

Exemplary Mechanism-Based Crosslinkers

In some embodiments, the mechanism-based crosslinker of the present invention has the formula:

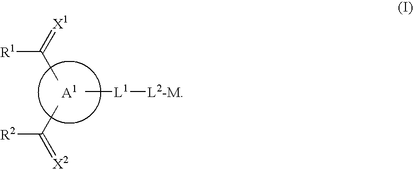

In Formula (I), X¹ and X² are independently O, S, or N. M is an ATP-binding moiety, as described above.

R¹ and R² are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A¹ is substituted or unsubstituted cycloalkyl, substituted or unsubstituted heterocycloalkyl, substituted or unsubstituted aryl, substituted or unsubstituted heteroaryl, or substituted or unsubstituted fused ring. Where A¹ is “substituted,” A¹ includes the substituents explicitly shown in Formula (I) (i.e. —C(X¹)R¹/—C(X²)R²) and additional substituents. Where A¹ is “unsubstituted,” A¹ includes only the substituents explicitly shown in Formula (I) (i.e. —C(X¹)R¹ and —C(X²)R²).

L² is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L¹ is a bond, —C(O)-L³-, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L³ is a bond, —O-L⁴-, or —N(R⁵)-L⁴-. R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. L⁴ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In some embodiments, X¹ and X² are O. In some related embodiments, R¹ and R² are hydrogen.

R¹ and R² may independently be hydrogen, halogen, substituted or unsubstituted C₁-C₂₀ alkyl, substituted or unsubstituted 2 to 20 membered heteroalkyl, C₃-C₈ substituted or unsubstituted cycloalkyl, 3 to 8 membered substituted or unsubstituted heterocycloalkyl, substituted or unsubstituted aryl, or substituted or unsubstituted heteroaryl.

R¹ and R² may also independently be hydrogen, halogen, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl. In some embodiments, R¹ and R² are hydrogen.

A¹ may be substituted or unsubstituted aryl, substituted or unsubstituted heteroaryl, or substituted or unsubstituted fused ring. In some embodiments, A¹ is unsubstituted aryl, unsubstituted heteroaryl, or unsubstituted fused ring. In other embodiments, A¹ is unsubstituted aryl, or unsubstituted heteroaryl (e.g. substituted or unsubstituted phenyl, substituted or unsubstituted pyridinyl, substituted or unsubstituted pyrazinyl and the like). In some embodiments, A¹ is a substituted or unsubstituted fused ring phenyl, such as quinolinyl, isoquinolinyl, benzofuranyl, indolyl, benzothiophenyl, carbazolyl, benzoimidazolyl, benzoxazolyl, benzothiazolyl, benzoisoxazolyl, benzopyrazolyl, benzoisothiazolyl, or naphthalenyl. A¹ may also be substituted or unsubstituted phenyl or substituted or unsubstituted naphthalenyl. In some embodiments, A¹ is unsubstituted phenyl or unsubstituted naphthalenyl.

L² may be a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene. L² may also be substituted or unsubstituted C₃-C₈ cycloalkylene, substituted or unsubstituted 3 to 8 membered heterocycloalkylene, substituted or unsubstituted arylene, or substituted or unsubstituted heteroarylene.

In some embodiments, L² is unsubstituted C₅-C₈ cycloalkylene; unsubstituted 5 to 8 membered heterocycloalkylene; unsubstituted arylene; unsubstituted heteroarylene; or C₅-C₈ cycloalkylene, 5 to 8 membered heterocycloalkylene, arylene, or heteroarylene substituted with at least one of the following groups: oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene.

In other embodiments, L² is unsubstituted C₅-C₈ cycloalkylene; unsubstituted 5 to 8 membered heterocycloalkylene; or C₅-C₈ cycloalkylene or 5 to 8 membered heterocycloalkylene substituted with one of the following groups: oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene.

L² may also be an unsubstituted 5 to 8 membered heterocycloalkylene; or C₅-C₈ cycloalkylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene.

Alternatively, L² is C₅-C₈ cycloalkylene substituted with an oxo, —OH, —NH₂, —CN, or halogen. L² may also simply be a ribose ring or deoxyribose ring.

In some embodiments, L¹ is a bond, —C(O)-L³-, —O—, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene. L³ is a bond, —O-L⁴-, —O—, or —N(R⁵)-L⁴-. R⁵ is hydrogen, substituted or unsubstituted C₁-C₂₀ alkyl, substituted or unsubstituted 2 to 20 membered heteroalkyl, C₃-C₈ substituted or unsubstituted cycloalkyl, 3 to 8 membered substituted or unsubstituted heterocycloalkyl, substituted or unsubstituted aryl, or substituted or unsubstituted heteroaryl. L⁴ is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene.

In other embodiments, L¹ may also be —C(O)-L³-, —O—, substituted or unsubstituted C₁-C₂₀ alkylene, substituted or unsubstituted 2 to 20 membered heteroalkylene. Alternatively, L¹ is a bond, —C(O)-L³-, —O—, unsubstituted C₁-C₂₀ alkylene, unsubstituted 2 to 20 membered heteroalkylene, unsubstituted C₃-C₈ cycloalkylene, unsubstituted 3 to 8 membered heterocycloalkylene, unsubstituted arylene, or unsubstituted heteroarylene. L¹ may also be —C(O)-L³-, —O—, unsubstituted C₁-C₂₀ alkylene, or unsubstituted heteroalkylene. In another embodiment, L¹ is a bond, —C(O)-L³-, unsubstituted C₁-C₁₀ alkylene, or unsubstituted 2 to 10 membered heteroalkylene. In yet another embodiment, L¹ is —C(O)—O—(CH₂)ₙ— or, —C(O)—NH—(CH₂)ₙ— or —(CH₂)ₙ—O—(CH₂)ₙ—, where n is an integer from 0 to 10. Alternatively n is an integer selected from 0, 1, 2, 3, 4, or 5.

R⁵ may be hydrogen, substituted or unsubstituted C₁-C₂₀ alkyl, or substituted or unsubstituted 2 to 20 membered heteroalkyl. In another embodiment, R⁵ is hydrogen, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl. Alternatively, R⁵ is hydrogen, unsubstituted C₁-C₂₀ alkyl, or unsubstituted heteroalkyl. R⁵ may also be hydrogen, or unsubstituted 2 to 20 membered heteroalkylene. In some embodiments, R⁵ is hydrogen.

In some embodiments, L⁴ is a bond, substituted or unsubstituted C₁-C₂₀ alkylene, or substituted or unsubstituted 2 to 20 membered heteroalkylene. In other embodiments, L⁴ is a bond, unsubstituted C₁-C₂₀ alkylene, unsubstituted 2 to 20 membered heteroalkylene, unsubstituted C₃-C₈ cycloalkylene, unsubstituted 3 to 8 membered heterocycloalkylene, unsubstituted arylene, or unsubstituted heteroarylene. L⁴ may be a bond, unsubstituted C₁-C₂₀ alkylene, or unsubstituted 2 to 20 membered heteroalkylene. L⁴ may also be unsubstituted C₁-C₁₀ alkylene. Alternatively, L⁴ is unsubstituted C₁-C₄ alkylene.

In some embodiments, the ATP-binding moiety has the formula:

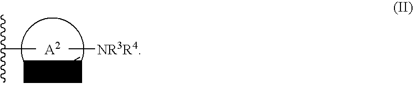

In Formula (II), A², R³, and R⁴ are as defined in the discussion of the compounds of Formula (III) below.

Thus, in some embodiments, the mechanism-based crosslinker has the formula:

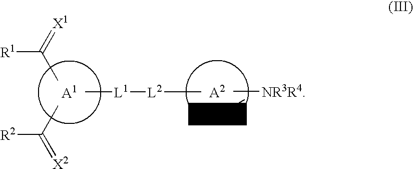

In Formula (III), R¹, R², A¹, X¹, X², L¹, and L² are as described above in the discussion of Formula (I).

R³ and R⁴ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In some embodiments, R³ and R⁴ are independently hydrogen, substituted or unsubstituted C₁-C₂₀ alkyl, substituted or unsubstituted 2 to 20 membered heteroalkyl, C₃-C₈ substituted or unsubstituted cycloalkyl, 3 to 8 membered substituted or unsubstituted heterocycloalkyl, substituted or unsubstituted aryl, or substituted or unsubstituted heteroaryl. In other embodiments, R³ and R⁴ are independently hydrogen, substituted or unsubstituted C₁-C₈ alkyl, substituted or unsubstituted aryl, or substituted or unsubstituted heteroaryl. In other embodiments, R³ is hydrogen and R⁴ is hydrogen, substituted or unsubstituted C₁-C₈ alkyl, substituted or unsubstituted aryl, or substituted or unsubstituted heteroaryl. In other embodiments, R³ is hydrogen and R⁴ is hydrogen. In other embodiments, R³ is hydrogen and R⁴ is substituted or unsubstituted heteroaryl.

A² is substituted or unsubstituted heterocycloalkyl, substituted or unsubstituted heteroaryl, or substituted or unsubstituted fused ring. Where A² is “substituted,” A² includes —NR³R⁴ and additional substituents. Where A² is “unsubstituted,” A² includes only —NR³R⁴.

In some embodiments, A² may substituted or unsubstituted fused ring. A² may also be unsubstituted fused ring. In some embodiments, A² is substituted or unsubstituted fused ring aryl. In other embodiments, A² is substituted or unsubstituted phenyl, substituted or unsubstituted purinyl, substituted or unsubstituted pyrimidinyl, substituted or unsubstituted pyridinyl, substituted or unsubstituted pyrazinyl, or substituted or unsubstituted pyridazinyl. In other embodiments, A² is unsubstituted purinyl.

In some embodiments of the compound of Formula (I) and/or (III), X¹ and X² are and R¹ and R² are hydrogen.

In other embodiments of the compound of Formula (I), L² is a bond. A¹ may be a substituted or unsubstituted aryl, or substituted or unsubstituted heteroaryl. M may have the formula:

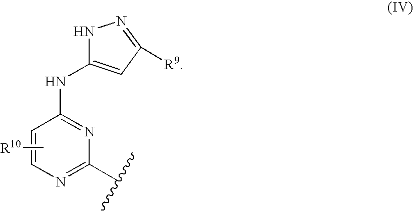

In Formula (IV), R⁹ and R¹⁰ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁹ is selected from hydrogen, substituted or unsubstituted C₁-C₂₀ alkyl, substituted or unsubstituted 2 to 20 membered heteroalkyl, C₃-C₈ substituted or unsubstituted cycloalkyl, 3 to 8 membered substituted or unsubstituted heterocycloalkyl, substituted or unsubstituted aryl, or substituted or unsubstituted heteroaryl. In other embodiments, R⁹ is selected from C₃-C₈ substituted or unsubstituted cycloalkyl, 3 to 8 membered substituted or unsubstituted heterocycloalkyl, substituted or unsubstituted aryl, or substituted or unsubstituted heteroaryl. In other embodiments, R⁹ is selected from C₃-C₈ substituted or unsubstituted cycloalkyl. In other embodiments, R⁹ is selected from C₃-C₈ unsubstituted cycloalkyl.

In some embodiments of the compound of Formula (IV), R¹⁰ is selected from hydrogen, substituted or unsubstituted C₁-C₂₀ alkyl, substituted or unsubstituted 2 to 20 membered heteroalkyl, C₃-C₈ substituted or unsubstituted cycloalkyl, 3 to 8 membered substituted or unsubstituted heterocycloalkyl, substituted or unsubstituted aryl, or substituted or unsubstituted heteroaryl. In other embodiments, R¹⁰ is selected from hydrogen and substituted or unsubstituted C₁-C₂₀ alkyl. In other embodiments, R¹⁰ is selected from hydrogen and unsubstituted C₁-C₂₀ alkyl. In other embodiments, R¹⁰ is hydrogen.

In some embodiments of the compound of Formula (IV), A¹ is substituted or unsubstituted phenyl or substituted or unsubstituted fused ring phenyl, such as substituted or unsubstituted benzoimidazolyl, or substituted or unsubstituted naphthalenyl. Thus, in some embodiments, the mechanism-based crosslinker has the formula:

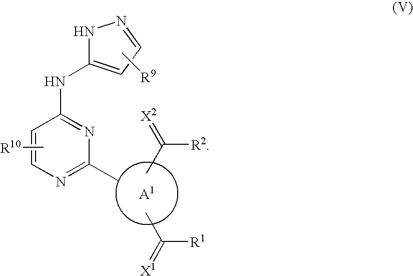

In Formula (V), X¹, X², R¹, R², and A¹ are as defined in the discussion of Formula (I), and R⁹ and R¹⁰ are as described above in the discussion of Formula (IV). In some embodiments, R⁹ is attached at the pyrazole 3 position.

In another embodiment, the mechanism-based crosslinker of the present invention has the formula:

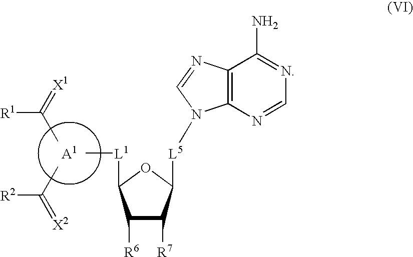

In Formula (VI), R¹, R², A¹, X¹, X², and L¹ are as described above in the discussion of Formula (I). In some embodiments of the compound of Formula (VI), X¹ and X² are O and R¹ and R² are hydrogen.

R⁶ and R⁷ are, independently, hydrogen, halogen, —OH, or —OR⁸. R⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. In some embodiments, R⁸ is substituted or unsubstituted alkyl, substituted or unsubstituted heteroalkyl. In another embodiment, R⁸ is substituted or unsubstituted C₁-C₂₀ alkyl, substituted or unsubstituted 2 to 20 membered heteroalkyl. R⁸ may be unsubstituted C₁-C₂₀ alkyl, or unsubstituted 2 to 20 membered heteroalkyl. R⁸ may be unsubstituted C₁-C₅ alkyl, or unsubstituted 2 to 5 membered heteroalkyl.

In some embodiments, R⁶ and R⁷ are —OH. In other embodiments, R⁶ is —OH and R⁷ is hydrogen.

L⁵ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

L⁵ may be a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene. L⁵ may also be a bond, unsubstituted C₁-C₂₀ alkylene, unsubstituted 2 to 20 membered heteroalkylene, unsubstituted C₃-C₈ cycloalkylene, unsubstituted 3 to 8 membered heterocycloalkylene, unsubstituted arylene, or substituted or unsubstituted heteroarylene.

In some embodiments, L⁵ is a bond, unsubstituted C₁-C₈ alkylene, or unsubstituted 2 to 8 membered heteroalkylene. Alternatively, L⁵ is a bond.

In another embodiment, the mechanism-based crosslinker of the present invention has the formula:

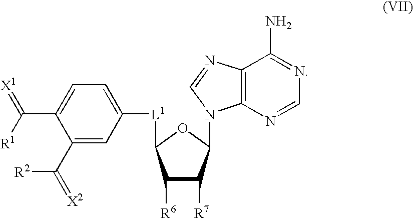

In Formula (VII), R¹, R², X¹, X², and L¹ are as described above in the discussion of Formula (I). R⁶ and R⁷ are as defined above in the discussion of Formula (VI). In some embodiments of the compound of Formula (IV), X¹ and X² are 0; R¹ and R² are hydrogen, R⁶ is —OH; R⁷ is —OH or hydrogen; and L¹ is —C(O)—O—CH₂— or, —C(O)—NH—CH₂— or —CH₂—O—CH₂—.

In another embodiment, the mechanism-based crosslinker of the present invention has the formula:

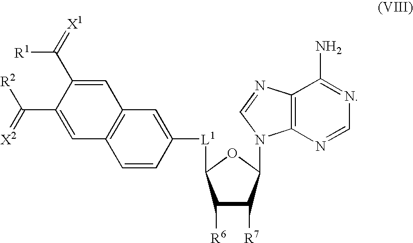

In Formula (VIII), R¹, R², X¹, X², and L¹ are as described above in the discussion of Formula (I). R⁶ and R⁷ are as defined above in the discussion of Formula (VI). In some embodiments of the compound of Formula (V), X¹ and X² are O; R¹ and R² are hydrogen, R⁶ is —OH; R⁷ is —OH or hydrogen; and L¹ is —C(O)—O—CH₂— or, —C(O)—NH—CH₂— or —CH2-O—CH₂—.

In some embodiments, each substituted group described above in the compounds of Formulae (I)-(VIII) is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, described above in the compounds of Formulae (I)-(VIII) are substituted with at least one substituent group. In other embodiments, at least one or all of these groups are substituted with at least one size-limited group. Alternatively, at least one or all of these groups are substituted with at least one lower substituent group.

In other embodiments of the compounds of Formulae (I)-(VIII), each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, each substituted or unsubstituted heterocycloalkyl is a substituted or unsubstituted 4 to 8 membered heterocycloalkyl, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₄-C₈ cycloalkylene, and/or each substituted or unsubstituted heterocycloalkylene is a substituted or unsubstituted 4 to 8 membered heterocycloalkylene.

Alternatively, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, each substituted or unsubstituted heterocycloalkyl is a substituted or unsubstituted 5 to 7 membered heterocycloalkyl, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₅-C₇ cycloalkylene, each substituted or unsubstituted heterocycloalkylene is a substituted or unsubstituted 5 to 7 membered heterocycloalkylene.

### II. Exemplary Syntheses

The compounds of the invention are synthesized by an appropriate combination of generally well known synthetic methods. Techniques useful in synthesizing the compounds of the invention are both readily apparent and accessible to those of skill in the relevant art. The discussion below is offered to illustrate certain of the diverse methods available for use in assembling the compounds of the invention. However, the discussion is not intended to define the scope of reactions or reaction sequences that are useful in preparing the compounds of the present invention.

Generally, mechanism-based crosslinkers can be generated by forming a single bond between an amine or oxygen of a heterocyclic kinase inhibitor and the carbonyl component (typically, a carboxylic acid, carboxylic acid chloride, or an activated ester of a carboxylic acid) or alkyl halide of the reactive moiety (Scheme 1). In addition, mechanism-based crosslinkers can be generated by forming a single bond between an amine or oxygen of the reactive moiety and the carbonyl component (typically, a carboxylic acid, carboxylic acid chloride, or an activated ester of a carboxylic acid) or alkyl halide of a heterocyclic kinase inhibitor (Scheme 2).

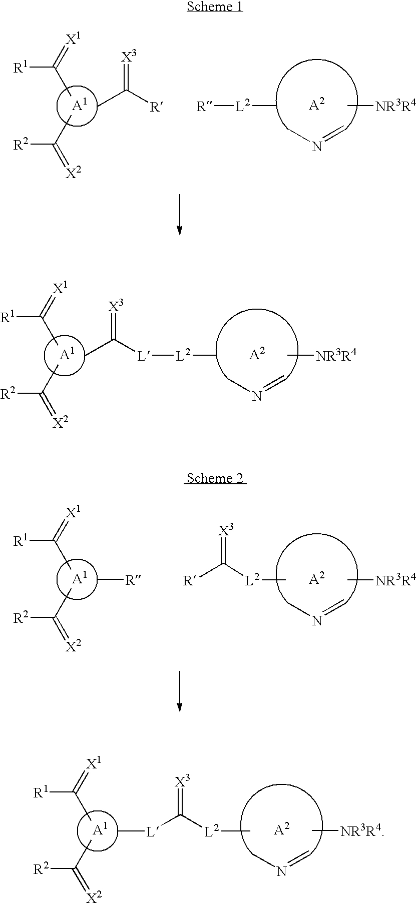

In Schemes 1 and 2, R¹, R², R³, R⁴, A¹, A², X¹, X², L¹ and L² are as described above in the discussion of Formula (I). X³ is selected from O, N, and S. R′ is —OH, substituted or unsubstituted alkoxy, or halogen. R″ is hydroxy or amino. L′ is the covalent linker product of R′ and R″.

### III. Kinases

The protein kinase family is one of the largest in the human genome, comprising some 500 genes (Manning et al., Science, 298, 1912 (2002); Kostich et al., Genome Biology, 3, research 0043.1 (2002)). The majority of kinases contain a 250-300 amino acid residue catalytic domain with a conserved core structure. This domain includes an ATP binding pocket (less frequently a GTP binding pocket). The phosphate donor is typically bound as a complex with a divalent ion (usually Mg²⁺ or Mn²⁺). Another important function of the catalytic domain is the binding and orientation for phosphotransfer of the substrate. The catalytic domains present in various kinases are largely homologous.

Significant progress has been made in determining the three dimensional structures of many kinases (Williams, D. H. and Mitchell, T., Curr. Opin. Pharmacol., 2, 567 (2002)). In addition, reliable homology models can be deduced and used successfully in structure-guided design. In fact, kinases have been classified into target family groups based on structure-activity relationships (SARs) of interactor chemotype series (Frye, S. V., Chem. Biol., 6, R3 (1999); Naumann, T. and Matter, H., J. Med. Chem., 45, 2366 (2002)).

Three motifs within the catalytic domain are thought to be critical for catalytic function, each of which contains an almost invariant residue believed to participate in catalysis (Manning et al., Science, 298: 1912-1934 (2002)). The motifs include: the VAIK motif (a catalytic lysine); the HRD motif (having a catalytic aspartate); and the DFG motif (D chelates Mg++ ions of ATP). It has been well documented that a single catalytic lysine residue is involved in the enzymatic mechanism of almost all known kinases (Kamps et al. Nature, 310, 589-592 (1984).

For example, the ATP analogue p-fluorosulphonylbenzoyl 5′-adenosine (FSBA) inactivates the tyrosine protein kinase activity of p60^{src} by reacting with lysine 295. FSBA is also known to react specifically with the ATP-binding site of cyclic AMP-dependent protein kinase and to modify lysine 71 (Zoller et al., J. Biol. Chem., 256, 10837-10842 (1981)). In addition, FSBA reacts with a homologous lysine residue in the cyclic GMP-dependent protein kinase, which has 42% sequence homology with the cyclic AMP-dependent protein kinase within this region (Hashimoto et al., J. biol. Chem., 257, 727-733 (1982)). Lysine 295 of p60^{src} aligns precisely with the reactive lysines found in these cyclic nucleotide-dependent protein kinases. Therefore, the tertiary structures of the ATP-binding regions of both cyclic nucleotide-dependent seline kinases and the tyrosine kinase p60^{src} all position a homologous lysine residue such that it reacts with FSBA.

In addition, yes (Kitamura et al., Nature, 297, 205-208 (1982)), fps (Shibuya et al., Cell, 30, 787-795 (1982)), abl (Reddy et al., Proc. Natl. Acad. Sci. U.S.A., 80, 3623-3627 (1983)), fgr Naharro et al., Science, 223, 63-66 (1984)) and erbB (Yamamoto et al., Cell, 35, 71-78 (1983)) each contains a lysine residue at a position homologous to lysine 295 of p60^{src}. In these proteins, a cluster of glycines with the sequence Gly-X-Gly-X-X-Gly lies 16-28 residues to the amino-terminal side of the lysines implicated in binding ATP (Sefton, B. M. and Hunter, T., Adv. Cyclic Nucleotide Protein Phosphorylation Res., 18, 195-226 (1984)).

In some embodiments, the kinase is selected from serine/threonine protein kinase A, Map Kinase p38, Casein kinase II, AKT1 kinase, and tyrosine kinase.

### IV. Methods

In another aspect, the present invention provides a method of detecting binding between an interactor and a kinase. The method includes contacting a kinase with a mechanism-based crosslinker and an interactor. The mechanism-based crosslinker is allowed to form a covalent bond with the interactor. The mechanism-based crosslinker is also allowed to specifically form a covalent bond with a catalytic amino acid side chain of the kinase thereby forming a crosslinked kinase-interactor pair. The presence of the crosslinked kinase-interactor pair is then detected, thereby detecting the binding between the interactor and the kinase.

In some embodiments, the covalent bond to the catalytic amino acid is formed only in the presence of the interactor. In another embodiment, the interactor binds to the peptide binding groove of the kinase.

Interactor reactive groups, catalytic amino acid reactive groups, and crosslinker reactive groups are described above and are equally applicable in the methods of the present invention. For example, the interactor may include an interactor reactive group. In some embodiments, the interactor reactive group is a thiol moiety that forms a covalent bond with the mechanism-based crosslinker. In addition, the catalytic amino acid side chain of the kinase may be a lysine amino acid side chain of the kinase. The mechanism-based crosslinker may include a first crosslinker reactive group and a second crosslinker reactive group. The first crosslinker reactive group and the second crosslinker reactive group may both be an aldehyde.

Mechanism-based crosslinkers are also described above and are equally applicable to the present methods. As described above, the mechanism-based crosslinker typically binds to the ATP-binding pocket of the kinase. Thus, the mechanism-based crosslinker may include an ATP-binding moiety.

Detection of the crosslinked kinase-interactor pair may be accomplished using any appropriate detection methodology. Exemplary detection methodologies include the use of standard protein purification methods, such as salt precipitation and solvent precipitation; methods utilizing the difference in molecular weight such as dialysis, ultra-filtration, gel-filtration, and SDS-polyacrylamide gel electrophoresis; methods utilizing a difference in electrical charge such as ion-exchange column chromatography, methods utilizing specific affinity such as affinity chromatography; methods utilizing a difference in hydrophobicity such as reverse-phase high performance liquid chromatography; and methods utilizing a difference in isoelectric point, such as isoelectric focusing electrophoresis.

Visualization and/or quantification of the isolated or non-isolated crosslinked kinase-interactor pair may be accomplished using any appropriate technique, including the use of dyes (e.g. protein dyes such as Commassie Blue) or detectable labels known in the art.

Detectable labels may be attached directly to the kinase, mechanism-based crosslinker, or interactor. Alternatively, the detectable label may be attached to a molecule that binds to the kinase, mechanism-based crosslinker, or interactor. The molecule that binds to the kinase, mechanism-based crosslinker, or interactor may also be referred to herein as a detector molecule. Detector molecules include any appropriate binding molecule, such as antibodies and affinity tag binders. Typically, where the detector molecule includes an affinity tag binder, the kinase, mechanism-based crosslinker, or interactor will include an affinity tag. Affinity tags are well known in the art and include, for example, T7 tag, S tag, His tag, GST tag, PKA tag, HA tag, c-Myc tag, Trx tag, Hsv tag, CBD tag, Dsb tag, pelB/ompT, KSI, MBP tag, VSV-G tag, β-Gal tag, GFP tag, V5 epitope tag, and FLAG epitope tag (Eastman Kodak Co., Rochester, N.Y.). In some embodiments, the interactor includes an affinity tag that binds to a detector molecule comprising an affinity tag binder.

Any appropriate detectable label is useful in the current invention, including, for example, luminescent labels, radioactive isotopic labels, enzymatic labels, and other labels well known in the art. Useful labels may be detectable by spectroscopic, photochemical, biochemical, immunochemical, electrical, magnetic, electromagnetic, optical or chemical means. Exemplary labels include magnetic bead labels (e.g., Dynabeads™); fluorescent dye labels (e.g., fluorescein isothiocyanate, texas red, rhodamine, green fluorescent protein, and the like); radiolabels (e.g., H³, I¹²⁵, S³⁵, C¹⁴, or P³²); enzyme labels (e.g., horse radish peroxidase, alkaline phosphatase and others commonly used in an ELISA); colorimetric labels such as colloidal gold, silver, selenium, or other metals and metal sol labels (see U.S. Pat. No. 5,120,643, which is herein incorporated by reference in its entirety for all purposes), or colored glass or plastic (e.g., polystyrene, polypropylene, latex, etc.) bead labels; and carbon black labels. Patents teaching the use of such detectable labels include U.S. Pat. Nos. 3,817,837; 3,850,752; 3,939,350; 3,996,345; 4,277,437; 4,275,149; 4,366,241; 6,312,914; 5,990,479; 6,207,392; 6,423,551; 6,251,303; 6,306,610; 6,322,901; 6,319,426; 6,326,144; and 6,444,143, which are herein incorporated by reference in their entirety for all purposes.

A variety of fluorescent detactable labels may be employed. Many such labels are commercially available from, for example, the SIGMA chemical company (Saint Louis, Mo.), Molecular Probes (Eugene, Oreg.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersburg, Md.), Fluka Chemica-Biochemika Analytika (Fluka Chemie AG, Buchs, Switzerland), and Applied Biosystems (Foster City, Calif.), as well as many other commercial sources known to one of skill. Furthermore, those of skill in the art will recognize how to select an appropriate fluorophore for a particular application and, if it not readily available commercially, will be able to synthesize the necessary fluorophore de novo or synthetically modify commercially available fluorescent compounds to arrive at the desired fluorescent label.

Means of detecting such labels are well known to those of skill in the art. Thus, for example, radiolabels may be detected using photographic film or scintillation counters, and fluorescent markers may be detected using a photodetector to detect emitted illumination. Enzymatic labels are typically detected by providing the enzyme with a substrate and detecting the reaction product produced by the action of the enzyme on the substrate, and calorimetric labels are detected by simply visualizing the colored label.

Detectable labels may be associated with the interactor, mechanism-based crosslinker, kinase, or detector molecule by any appropriate means, including, for example, covalent bonding, hydrogen bonding, van der Waal forces, π bond stacking, hydrophobic interactions, and ionic bonding.

The detectable labels may be covalently attached to the interactor, mechanism-based crosslinker, kinase, or detector molecule using a reactive functional group, which can be located at any appropriate position. When the reactive group is attached to an alkyl, or substituted alkyl chain tethered to an aryl nucleus, the reactive group may be located at a terminal position of an alkyl chain. Reactive groups and classes of reactions useful in practicing the present invention are generally those that are well known in the art of bioconjugate chemistry. Currently favored classes of reactions available with reactive known reactive groups are those which proceed under relatively mild conditions. These include, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

Useful reactive functional groups include, for example:

(a) carboxyl groups and various derivatives thereof including, but not limited to, N-hydroxysuccinimide esters, N-hydroxybenzotriazole esters, acid halides, acyl imidazoles, thioesters, p-nitrophenyl esters, alkyl, alkenyl, alkynyl and aromatic esters;

(b) hydroxyl groups which can be converted to esters, ethers, aldehydes, etc.

(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;

(d) dienophile groups which are capable of participating in Diels-Alder reactions such as, for example, maleimido groups;

(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;

(f) sulfonyl halide groups for subsequent reaction with amines, for example, to form sulfonamides;

(g) thiol groups, which can be converted to disulfides or reacted with acyl halides;

(h) amine or sulfhydryl groups, which can be, for example, acylated, alkylated or oxidized;

(i) alkenes, which can undergo, for example, cycloadditions, acylation, Michael addition, etc;

(j) epoxides, which can react with, for example, amines and hydroxyl compounds; and

(k) phosphoramidites and other standard functional groups useful in nucleic acid synthesis.

The reactive functional groups can be chosen such that they do not participate in, or interfere with, the crosslinking reactions disclosed herein. Alternatively, a reactive functional group can be protected from participating in the crosslinking reaction by the presence of a protecting group. Those of skill in the art will understand how to protect a particular functional group from interfering with a chosen set of reaction conditions. For examples of useful protecting groups, See Greene et al., PROTECTIVE GROUPS IN ORGANIC SYNTHESIS, John Wiley & Sons, New York, 1991.

Linkers may also be employed to attach the detectable labels to the interactor, mechanism-based crosslinker, kinase, or detector molecule. Linkers may include reactive groups at the point of attachment to the detectable label and/or the mobile detectable analyte binding reagents. Any appropriate linker may be used in the present invention, including substituted or unsubstituted alkylene, substituted or unsubstituted heteroalkylene, substituted or unsubstituted cycloalkylene, substituted or unsubstituted heterocycloalkylene, substituted or unsubstituted arylene, and substituted or unsubstituted heteroarylene. Other useful linkers include those having a polyester backbone (e.g. polyethylene glycol), nucleic acid backbones, amino acid backbones, and derivatives thereof. A wide variety of useful linkers are commercially available (e.g. polyethylene glycol based linkers such as those available from Nektar, Inc. of Huntsville, Ala.).

The detectable label may also be non-covalently attached to the interactor, mechanism-based crosslinker, kinase, or detector molecule any appropriate binding pair (e.g. biotin-sterptaviding, his tags, and the like).

Alternatively, ellipsometry (see, e.g., Elwing, H. Biomaterials 19(4-5):397-406 (1998); Werner, C. et al. Int. J. Artif. Organs 22(3):160-176 (1999); and Ostroff, R M. et al. Clin. Chem. 45(9):1659-64 (1999)) or surface plasmon resonance spectroscopy (see e.g., Mrksich, M.; et al., Langmair 1995, 4383; Mrksich, M., et al., J. Am. Chem. Soc. 1995, 117:12009; Sigal, G. B., et al., Anal. Chem 1996, 68:490) can also be used to detect binding events (e.g., on surfaces).

In another embodiment, the crosslinked kinase-interactor pair is itself luminescent. In another embodiment, the crosslinked kinase-interactor pair is fluorescent.

In another aspect, the present invention provides a method of identifying an interactor of a kinase. The method includes contacting a kinase with a mechanism-based crosslinker and an interactor. The mechanism-based crosslinker is allowed to form a covalent bond with the interactor. The mechanism-based crosslinker is also allowed to specifically form a covalent bond with a catalytic amino acid side chain of the kinase thereby forming a crosslinked kinase-interactor pair. The presence of the crosslinked kinase-interactor pair is then detected, thereby identifying the interactor of the kinase.

### V. Arrays

In another aspect, the present invention provides a method of detecting an active kinase in a sample. The method includes contacting an immobilized interactor with a mechanism-based crosslinker and a sample comprising an active kinase. The method also includes allowing the mechanism-based crosslinker to form a covalent bond with the interactor and specifically form a covalent bond with a catalytic amino acid side chain of the active kinase. An immobilized crosslinked kinase-interactor pair is thereby formed. Finally, the presence of the immobilized crosslinked kinase-interactor pair is detected thereby detecting the active kinase.

In some embodiments, a plurality of interactors are immobilized in an array format. In this embodiment, a method is provided to detect an active kinase in a sample. The method includes contacting an array of immobilized interactors with a mechanism-based crosslinker and a sample comprising an active kinase. The method also includes allowing the mechanism-based crosslinker to form a covalent bond with an interactor and specifically form a covalent bond with a catalytic amino acid side chain of the active kinase. An immobilized crosslinked kinase-interactor pair is thereby formed. Finally, the presence of the immobilized crosslinked kinase-interactor pair is detected thereby detecting the active kinase.

Interactors may be immobilized to a solid support using any appropriate conjugation technique (Hermanson, BIOCONJUGATE TECHNIQUES, Academic Press, San Diego, 1996). Useful reactive functional groups discussed above in the context of detectable label attachment is equally applicable for immobilizing interactors. By “immobilized” and grammatical equivalents herein is meant the association or binding between the interactor and the solid support is sufficient to be stable under the conditions of binding, washing, analysis, and removal as outlined below. The binding can be covalent or non-covalent. Included in non-covalent binding is the covalent attachment of a molecule, such as, streptavidin to the support and the non-covalent binding of the biotinylated probe to the streptavidin. Covalent bonds can be formed directly between the interactor and the solid support or can be formed by a linker or by inclusion of a functional reactive group on either the solid support or the probe or both molecules. Immobilization may also involve a combination of covalent and non-covalent interactions.

Interactors may be immobilized to any appropriate solid support known in the art. In some embodiments, the interactors are attached to a biochip. Biochips typically include a suitable solid substrate. By “substrate” or “solid support” or other grammatical equivalents herein is meant any material that can be modified to contain discrete individual sites appropriate for the attachment or association of the interactors and is amenable to at least one detection method. As will be appreciated by those in the art, the number of possible substrates are very large, and include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica-based materials including silicon and modified silicon, carbon, metals, inorganic glasses, plastics, etc. In general, the interactors allow optical detection and do not appreciably show fluorescence.

Interactors may be attached to solid supports in a wide variety of ways, as will be appreciated by those in the art. The interactors can either be synthesized first, with subsequent attachment to, for example, a biochip, or can be directly synthesized on the biochip.

In some embodiments, the surface of a biochip and the interactor may be derivatized with chemical functional groups such as those described above in the context of label attachment.

In some embodiments, the array of interactors is an array of identical interactors. In other embodiments, the array includes patches of different interactors.

In another aspect, the present invention provides an array of immobilized interactors crosslinked to a mechanism-based crosslinker and an active kinase, as described above.

The terms and expressions which have been employed herein are used as terms of description and not of limitation, and there is no intention in the use of such terms and expressions of excluding equivalents of the features shown and described, or portions thereof, it being recognized that various modifications are possible within the scope of the invention claimed. Moreover, any one or more features of any embodiment of the invention may be combined with any one or more other features of any other embodiment of the invention, without departing from the scope of the invention. For example, the features of the mechanism-based crosslinkers are equally applicable to the methods of detecting binding between an interactor and a kinase described herein. All publications, patents, and patent applications cited herein are hereby incorporated by reference in their entirety for all purposes.

### EXAMPLES

### Materials

Unless otherwise noted, all reagents were obtained from commercial suppliers and used without further purification. 2′,3′-isopropylideneadenosine, 3-carboxybenzaldehyde, 4-carboxybenzaldehyde, 1-(2-mesitylenesulfonyl)-3-nitro-1,2,4-triazole (MSNT), triisopropylsilane (TIS),1,2-ethanedithiol, 2-aminoethanol, β-mercaptoethanol (BME), diisopropylcarbodiimide (DIC), trifluoroacetic acid (TFA), biotin, ATP, and N-acetylcysteine were purchased from Aldrich. 6-carboxyfluorescein was purchased from molecular probes. Fmoc-amino acids, 1-hydroxybenzotriazole (HOBt), and N-α-Fmoc amino acids attached to Wang resin were purchased from Novabiochem. Anhydrous, low-amine N,N-dimethylformamide (DMF) was purchased from EM science. Fmoc-hCys(trt)-OH, Fmoc-Cys(Me)-OH, and Fmoc-Pen(trt)-OH were purchased from Bachem. Recombinant AKT1, Casein Kinase II, and cAMP-dependent Protein Kinase (PKA) were purchased from Calbiochem. The abbreviation “Ahx” refers to aminohexanoic acid (also referred to herein within the context of an amino acid sequence as “Z”).

### Example 1

### Synthesis of Dialdehyde

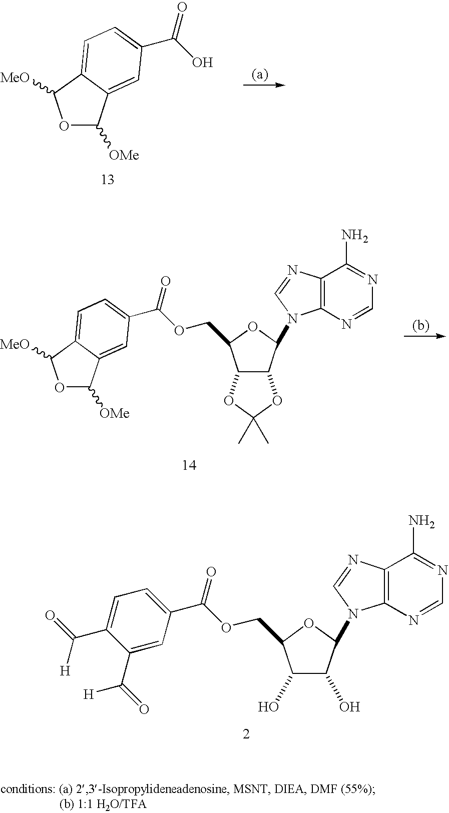

Bis-acetyl protected 3,4-diformylbenzoic 13 acid was prepared via a modified literature procedure (Le Bourdomiec et al., J. Med. Chem. 43, 2489-2492 (2000). To a 25 mL round bottom flask were added 2′,3′-isopropylideneadenosine (750 mg, 2.4 mmol), 13 (280 mg, 1.2 mmol), DIEA (0.42 mL, 2.5 mmol), and DMF (8 mL). The reaction mixture was cooled to 0° C. and MSNT (360 mg, 1.2 mmol) was added portion-wise. The reaction mixture was stirred for 12 h at rt and then concentrated in vacuo. The resultant red oil was dissolved in EtOAc (200 mL), extracted with 10% citric acid (100 mL), and 10% NaHCO₃ (100 mL). The organic layer was dried (Na₂SO₄) and concentrated. Purification over SiO₂ (5:95 MeOH/CHCl₃) provided 350 mg (56%) of bis-acetyl 14 as a mixture of diastereomers. ¹H-NMR (400 MHz, CDCl₃) δ 8.40-7.58 (m, 5H), 6.11-5.96 (m, 3H), 5.46 (m, 1H), 5.08 (m, 1H), 4.46-4.38 (m, 3H), 3.34-3.24 (m, 6H), 1.59 (s, 3H), 1.38 (s, 3H); MS (ESI), m/z calculated for C₂₄H₂₇N₅O₈: 513.2. Found: m/z 514.2 (M+H)+, 536.2 (M+Na)+. Bis-acetal 14 (15 mg, 29 μmol) was dissolved in 1:1 TFA/H2O (1 mL), stirred for 1 h at rt, and the TFA was removed in vacuo. The resultant aqueous solution was purified using c18 reverse-phase HPLC (CH₃CN/H₂O-No TFA). The purified product was concentrated by lyophilization to afford 5.5 mg (42%) of 2 as a white powder. ¹H-NMR (400 MHz, 1:1 DMSO-d6/H₂O-d2) δ 8.09 (m, 1H), 7.91 (s, 1H), 7.83-7.76 (m, 2H), 7.39 (m, 1H), 6.39 (m, 1H), 6.12 (m, 1H), 5.83 (m, 1H), 4.72 (m, 1H), 4.58 (m, 1H), 4.46-4.40 (m, 2H), 4.21 (m, 1H); MS (ESI), m/z calculated for C19H17N5O7: 427.1. Found: m/z 428.1 (M+H)⁺.

Bis-acetal 14 (15 mg, 29 μmol) was dissolved in 1:1 TFA/H₂O (1 mL), stirred for 1 h at rt, and the TFA was removed in vacuo. The resultant aqueous solution was purified using c18 reverse-phase HPLC (CH₃CN/H₂O-No TFA). The purified product was concentrated by lyophilization to afford 5.5 mg (42%) of 2 as a white powder. ¹H-NMR (400 MHz, 1:1 DMSO-d6/H2O-d2) δ 8.09 (m, 1H), 7.91 (s, 1H), 7.83-7.76 (m, 2H), 7.39 (m, 1H), 6.39 (m, 1H), 6.12 (m, 1H), 5.83 (m, 1H), 4.72 (m, 1H), 4.58 (m, 1H), 4.46-4.40 (m, 2H), 4.21 (m, 1H); MS (ESI), m/z calculated for C19H17N5O7: 427.1. Found: m/z 428.1 (M+H)+.

### Example 2

### Conversion of Dialdehyde to Isonindole

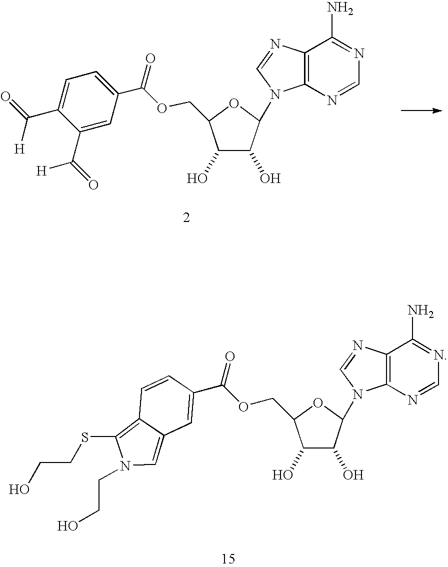

To lend further proof of identity, dialdehyde 2 was converted to isoindole 15. Dialdehyde 2 (3.5 mg, 8.2 μMol), BME (78 mg, 1.0 mMol), and 2-aminoethanol (61 mg, 1.0 mMol) were dissolved in 1:1 CH₃CN/H₂O (1 mL) and stirred at rt for 3 h. The reaction mixture was concentrated and purified using c18 reverse-phase HPLC (CH₃CN/H₂O-0.1% TFA). The purified product was concentrated by lyophilization to afford 1.2 mg (32%) of 15 as a yellow powder. MS (ESI), m/z calculated for C₂₁H₂ON₆O₅S: 468.1. Found: m/z 469.3 (M+H)⁺.

### Example 3

### Synthesis of 3-formylbenzoyladenosine

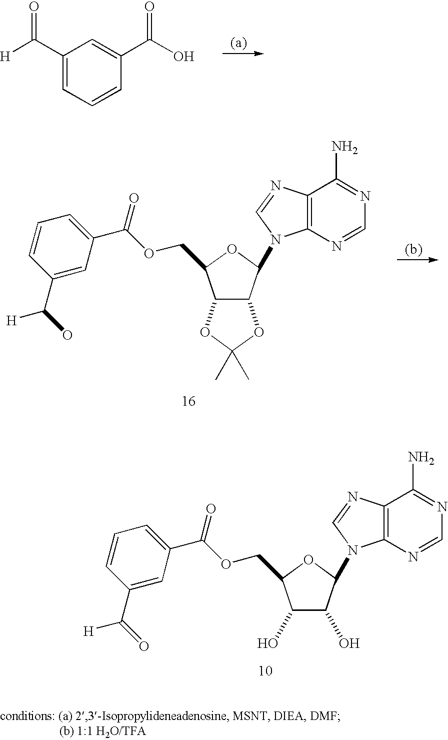

To a 10 mL round bottom flask were added 2′,3′-isopropylideneadenosine (200 mg, 0.65 mmol), 3-carboxybenzaldehyde (98 mg, 0.65 mmol), DIEA (0.23 mL, 1.3 mmol), and DMF (2 mL). The reaction mixture was cooled to 0° C. and MSNT (190 mg, 0.65 mmol) was added portion-wise. The reaction mixture was stirred for 12 h at rt and then concentrated in vacuo. The resultant yellow oil was dissolved in EtOAc (100 mL), extracted with 10% citric acid (50 mL), and 10% NaHCO₃ (50 mL). The organic layer was dried (Na₂SO₄) and concentrated. Purification over SiO₂ (5:95 MeOH/CHCl₃) provided 204 mg (71%) of 16 as a clear oil. ¹H-NMR (400 MHz, DMSO-d6) δ 10.00 (s, 1H), 8.29 (m, 1H), 8.26 (s, 1H), 8.21-8.06 (m, 3H), 7.56 (t, J=8.0 Hz, 1H), 7.28 (br s, 2H), 6.20 (m, 1H), 5.52 (m, 1H), 5.18 (m, 1H), 4.61-4.41 (m, 3H), 1.52 (s, 3H), 1.31 (s, 3H); MS (ESI), m/z calculated for C21H21N5O6: 439.2. Found: m/z 440.3 (M+H)+. 16 (53 mg, 0.12 mmol) was dissolved in 1:1 TFA/H₂O (3 mL), stirred for 1 h at rt, and the TFA was removed in vacuo. The resultant aqueous solution was then purified using c18 reverse-phase HPLC (CH₃CN/H₂O-0.1% TFA). The purified product was concentrated by lyophilization to afford 12 mg (25%) of 10 as a white powder. ¹H-NMR (400 MHz, DMSO-d6) δ 10.05 (s, 1H), 8.46 (m, 1H), 8.38 (s, 1H), 8.20 (br s, 2H), 8.19-8.13 (m, 3H), 7.72 (t, J=8.0 Hz, 1H), 5.94 (m, 1H), 5.49 (br m, 1H), 5.09 (br m, 1H), 4.69-4.61 (m, 2H), 4.51-4.47 (m, 1H), 4.40 (m, 1H), 4.24 (m, 1H); MS (ESI), m/z calculated for C18H17N5O6: 399.1. Found: m/z 400.2 (M+H)+.

### Example 4

### Synthesis of 4-formylbenzoyladenosine

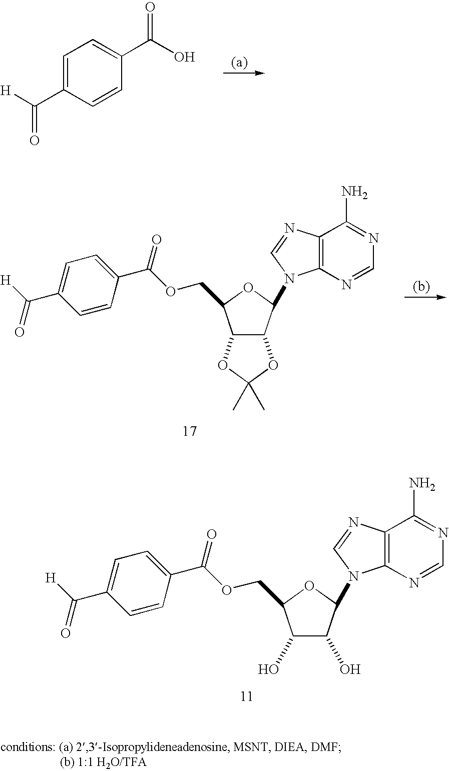

To a 10 mL round bottom flask were added 2′,3′-isopropylideneadenosine (200 mg, 0.65 mmol), 4-carboxybenzaldehyde (98 mg, 0.65 mmol), DIEA (0.23 mL, 1.3 mmol), and DMF (2 mL). The reaction mixture was cooled to 0° C. and MSNT (190 mg, 0.65 mmol) was added portion-wise. The reaction mixture was stirred for 12 h at rt and then concentrated in vacuo. The resultant yellow oil was dissolved in EtOAc (100 mL), extracted with 10% citric acid (50 mL), and 10% NaHCO₃ (50 mL). The organic layer was dried (Na₂SO₄) and concentrated. Purification over SiO₂ (5:95 MeOH/CHCl₃) provided 215 mg (75%) of 17 as a clear oil. ¹H-NMR (400 MHz, DMSO-d6) δ 10.06 (s, 1H), 8.26 (m, 1H), 8.08 (s, 1H), 8.02 (m, 2H), 7.95 (m, 2H), 7.35 (br s, 2H), 6.19 (m, 1H), 5.53 (m, 1H), 5.18 (m, 1H), 4.58-4.39 (m, 3H), 1.53 (s, 3H), 1.32 (s, 3H); MS (ESI), m/z calculated for C₂₁H₂₁N₅O₆: 439.2. Found: m/z 440.4 (M+H)+. 17 (47 mg, 0.11 mmol) was dissolved in 1:1 TFA/H₂O (3 mL), stirred for 1 h at rt, and the TFA was removed in vacuo. The resultant aqueous solution was then purified using c18 reverse-phase HPLC (CH₃CN/H₂O-0.1% TFA). The purified product was subsequently concentrated by lyophilization to afford 9 mg (21%) of 11 as a white powder. ¹H-NMR (400 MHz, DMSO-d6) δ 10.08 (s, 1H), 8.49 (m, 1H), 8.25 (s, 1H), 8.08 (m, 2H), 7.99 (m, 2H), 5.94 (m, 1H), 5.49 (br m, 1H), 5.09 (br m, 1H), 4.69-4.60 (m, 2H), 4.50-4.39 (m, 2H), 4.24 (m, 1H); MS (ESI), m/z calculated for C18H17N5O6: 399.1. Found: m/z 400.2 (M+H)+.

### Example 5

### General Peptide Synthesis Procedure

Fmoc-L-amino acid-Wang resin (0.10 g) and DMF were added to a 3 mL syringe cartridge was added. Synthesis was performed using standard DIC/HOBt activation of amino acids. The coupling reaction was performed for 4 h with a 0.4 M concentration of activated amino acid. The Fmoc-protecting group was removed after every step using 20% piperidine in DMF, followed by washing with DMF (3×). Prior to cleavage, the resin was washed with DMF (3×), CH₂Cl₂ (3×), MeOH (3×), and CH₂Cl₂ (3×). The peptide was cleaved from resin by treatment with 94:2:2:2 TFA/1,2-ethanedithiol/H₂O/TIS for 2 h. The solvent was removed in vacuo and the resulting crude product was purified by C18 reverse-phase HPLC (CH₃CN/H₂O/-0.1% TFA).

### Example 6

### Biotin-Ahx-Arg-Pro-Arg-Thr-Ser-Ser-Phe-OH (3)

The substrate was prepared according to the general peptide synthesis procedure using Fmoc-Phe-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₅₂H₈₄N₁₆O₁₄S: 1188.6. Found: m/z 1189.7 (M+H)+.

### Example 7

### Fluorescein-Ahx-Ahx-Arg-Pro-Arg-Thr-Ser-Ser-Phe-OH (4)

The substrate was prepared according to the general peptide synthesis procedure using Fmoc-Phe-Wang resin. The N-terminus was capped by agitating the resin overnight in the presence of 6-carboxyfluorescein (0.1 M), HOBt (0.1 M) and DIC (0.1 M). Following reverse-phase HPLC and lyophilization the peptide was obtained as a yellow solid. MS (ESI), m/z calculated for C₆₉H₉₁N₁₀O₁₉: 1433.7. Found: m/z 1434.8 (M+H)+.

### Example 8

### Biotin-Ahx-Arg-Pro-Arg-Thr-Ser-Cys-Phe-OH (5)

The substrate was prepared according to the general peptide synthesis procedure using Fmoc-Phe-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₅₂H₈₄N₁₆O₁₃S₂: 1204.6. Found: m/z 1205.7 (M+H)+.

### Example 9

### Fluorescein-Ahx-Ahx-Arg-Pro-Arg-Thr-Ser-Cys-Phe-OH (6)

The substrate was prepared according to the general peptide synthesis procedure using Fmoc-Phe-Wang resin. The N-terminus was capped by agitating the resin overnight in the presence of 6-carboxyfluorescein (0.1 M), HOBt (0.1 M) and DIC (0.1 M). Following reverse-phase HPLC and lyophilization the peptide was obtained as a yellow solid. MS (ESI), m/z calculated for C₆₉H₉₁N₁₅O₁₈S: 1449.6. Found: m/z 1450.8 (M+H)+.

### Example 10

### Biotin-Ahx-Arg-Pro-Arg-Thr-Ser-hCys-Phe-OH

The substrate was prepared according to the general peptide synthesis procedure using Fmoc-Phe-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₅₃H₈₆N₁₆O₁₃S₂: 1218.6. Found: m/z 1219.8 (M+H)+.

### Example 11

### Biotin-Ahx-Arg-Pro-Arg-Thr-Ser-Pen-Phe-OH

The substrate was prepared according to the general peptide synthesis procedure using Fmoc-Phe-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₅₄H₈₈N₁₆O₁₃S₂: 1232.6. Found: m/z 1233.9 (M+H)+.

### Example 12

### Biotin-Ahx-Arg-Pro-Arg-Thr-Ser-Cys(Me)-Phe-OH

The substrate was prepared according to the general peptide synthesis procedure using Fmoc-Phe-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₅₃H₈₆N₁₆O₁₃S₂: 1218.6. Found: m/z 1219.8 (M+H)+.

### Example 13

### Biotin-Ahx-Ile-Pro-Thr-Cys-Pro-Ile-Thr-Thr-Thr-Tyr-Phe-OH

The substrate was prepared according to the general peptide synthesis procedure using Fmoc-Phe-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₇₅H₁₁₄N₁₄O₂₀S₂: 1594.8. Found: m/z 1595.9 (M+H)+.

### Example 14

### Biotin-Ahx-Arg-Arg-Ala-Asp-Asp-Cys-Asp-Asp-Asp-Asp-OH

The substrate was prepared according to the general peptide synthesis procedure using Fmoc-Asp(O-t-Bu)-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₅₈H₉₁N₁₉O₂₆S₂ 1533.6. Found: m/z 768.0 (M+2H)2+.

### Example 15

### Biotin-Ahx-Leu-Arg-Arg-Ala-Cys-Leu-Gly-OH

The substrate was prepared according to the general peptide synthesis procedure using Fmoc-Gly-Wang resin. The N-terminus was capped by agitating the resin overnight in the presence of biotin (0.1 M), HOBt (0.1 M) and DIC (0.1 M). Following reverse-phase HPLC and lyophilization the peptide was obtained as a white solid. MS (ESI), m/z calculated for C₄₉H₈₆N₁₆O₁₁S₂: 1126.6. Found: m/z 1127.7 (M+H)+.

### Example 16

### Crosslinking Reactions with AKT1

His 6-tagged human Akt1 (10 ng), 1 μM peptide 3 or 4, and 20 M crosslinker 2 were incubated in 30 μL of 25 mM HEPES (pH=7.5), 150 mM NaCl, 2 mM MgCl2, 20 μM BME for 20 min at rt. After 20 min, 6 μL of 6× loading buffer was added to quench the reactions. The sample mixtures (10 μL) were resolved by 8-16% SDS-PAGE, transferred to nitrocellulose, and the membrane was probed with streptavidin-HRP.

### Example 17

### Crosslinking Reactions with p38

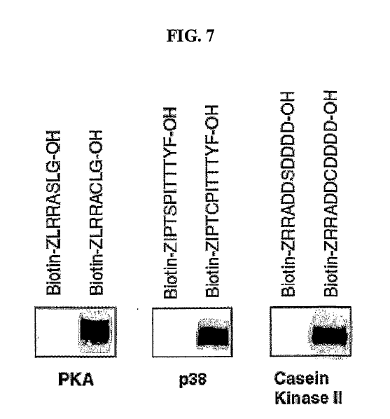

His6-tagged p38-as1 (600 ng), 20 μM Biotin-Ahx-Ile-Pro-Thr-Cys-Pro-Ile-Thr-Thr-Thr-Tyr-Phe-OH (X=Cys or Ser), and 20 μM crosslinker 2 were incubated in 30 μL of 25 mM HEPES (pH=7.5), 150 mM NaCl, 2 mM MgCl2, 100 μM BME for 1 h at rt. After 1 h, 6 μL of 6× loading buffer was added to quench the reactions. The sample mixtures (10 μL) were resolved by 8-16% SDS-PAGE, transferred to nitrocellulose, and the membrane was probed with streptavidin-HRP. Results are shown in FIG. 7.

### Example 18

### Crosslinking Reactions with Casein Kinase II

Casein Kinase II (200 ng), 20 μM Biotin-Ahx-Arg-Arg-Ala-Asp-Asp-X-Asp-Asp-Asp-Asp-OH (X=Cys or Ser), and 100 μM crosslinker 2 were incubated in 30 μL of 25 mM HEPES (pH=7.5), 150 mM NaCl, 2 mM MgCl₂, 100 μM BME for 1 h at rt. After 1 h, 6 μL of 6× loading buffer was added to quench the reactions. The sample mixtures (10 μL) were resolved by 8-16% SDS-PAGE, transferred to nitrocellulose, and the membrane was probed with streptavidin-HRP. Results are shown in FIG. 7.

### Example 19

### Crosslinking Reactions with PKA

PKA (200 ng), 5 μM Biotin-Ahx-Leu-Arg-Arg-Ala-X-Leu-Gly-OH (X=Cys or Ser) and 20 μM crosslinker 2 were incubated in 30 μL of 25 mM HEPES (pH=7.5), 150 mM NaCl, 2 mM MgCl2, 20 μM BME for 20 min at rt. After 20 min, 6 μL of 6× loading buffer was added to quench the reactions. The sample mixtures (10 μL) were resolved by 8-16% SDS-PAGE, transferred to nitrocellulose, and the membrane was probed with streptavidin-HRP. Results are shown in FIG. 7.

### Example 20

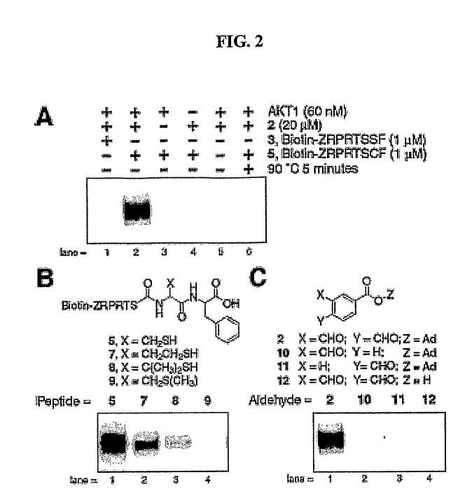

To evaluate dialdehyde 2 as a mechanism-based crosslinker, a set of peptide substrate derivatives with and without an engineered cysteine (biotin-ZRPRTSSF-OH 3, fluorescein-ZZRPRTSSF-OH 4, biotin-ZRPRTSCF-OH 5 and fluorescein-ZZRPRTSCF—OH 6) were incubated with the kinase, AKT1 (Alessi et al., FEBS Lett. 399: 333-338 (1996)). As shown in FIG. 2A, incubation of AKT1 and the serine-containing peptide 3 in the presence of dialdehyde crosslinker 2 led to no detectable crosslinking (lane 1). However, when cysteine-containing peptide 5 and AKT1 were incubated with dialdehyde 2, rapid formation of the peptide-AKT1 complex was observed (lane 2), demonstrating the necessity of a free thiol for isoindole formation. As expected, the peptide-AKT1 covalent complex runs at a slightly higher molecular weight than unmodified AKT1 on SDS-PAGE. All three components were found to be necessary, as labeling is not observed in the absence of dialdehyde 2 (lane 3), AKT1 (lane 4) or peptide 5 (lane 5). In addition, the absence of the peptide-AKT1 complex upon prior heat denaturation of AKT1 demonstrates that the protein must be properly folded for the crosslinking reaction to occur (lane 6). The crosslinking reaction is independent of the reporter group, as identical results were obtained for peptides 5 and 6.

### Example 21

Prior examples demonstrated the ability of the crosslinker to link a kinase and its substrate in a purified system. The following experiment demonstrates that this methodology can be carried out in the context of a complex biological mixture (cell lysate).

Kinase cell lysate crosslinking details: 1 μM of Akt substrate (Biotin-PEG-RPRTSCF-OH) and 100 ng of AKT kinase was added to 2 μg of HeLa cell lysate. 100 μM of the crosslinker 2 was added to cell lysate and the reaction was allowed to proceed for 90 minutes. The lysate were poured over avidin-agarose beads and the beads were washed with wash buffer. The bound protein was eluted from the agarose beads with loading buffer and then subjected to SDS-PAGE. The gel was transferred to nitrocellulose and probed with anti-AKT. A positive signal was observed. As a control, the non-cysteine containing AKT substrate (Biotin-PEG-RPRTSSF-OH) was used in the crosslinking reaction described above. No signal was observed.

### Example 22

Some compounds of the present invention include:

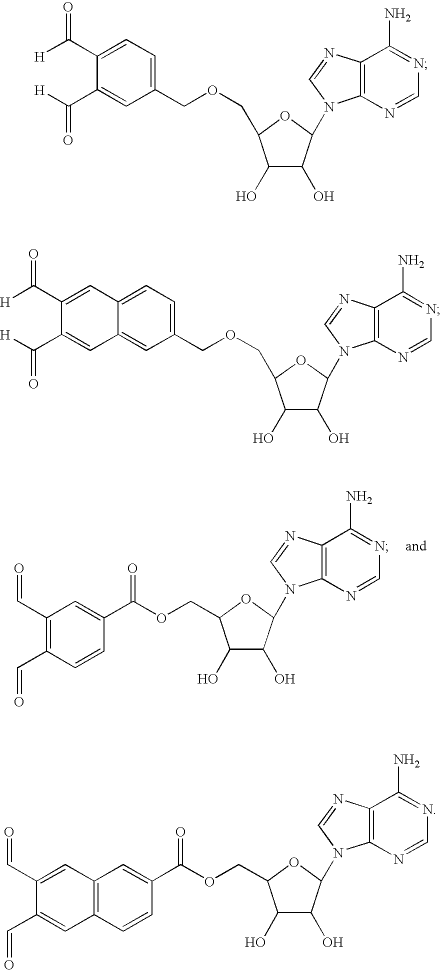

What is claimed is:

1. A compound having the formula: 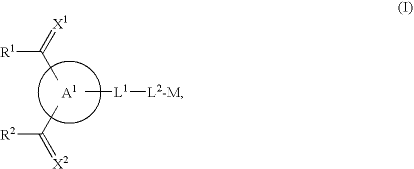
wherein,
X¹ and X² are independently O, S, or N;
R¹ and R² are independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
A¹ is substituted or unsubstituted cycloalkyl, substituted or unsubstituted heterocycloalkyl, substituted or unsubstituted aryl, substituted or unsubstituted heteroaryl, or substituted or unsubstituted fused ring;
L² is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene;
L¹ is a bond, —C(O)-L³-, —O—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, wherein
L³ is a bond, —O-L⁴-, or —N(R⁵)-L⁴-, wherein
R⁵ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl, and
L⁴ is a bond, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene, and
M is an ATP-binding moiety.

2. The compound of claim 1, wherein M has the formula 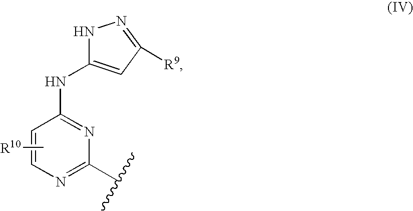
wherein,
R⁹ and R¹⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

3. The compound of claim 2, wherein R⁹ is C₃-C₈ substituted or unsubstituted cycloalkyl, 3 to 8 membered substituted or unsubstituted heterocycloalkyl, substituted or unsubstituted aryl, or substituted or unsubstituted heteroaryl.

4. The compound of claim 2, wherein R¹⁰ is hydrogen or substituted or unsubstituted C₁-C₂₀ alkyl.

5. The compound of claim 1, wherein M has the formula: 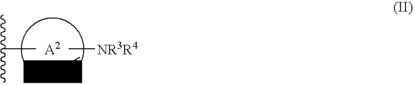
wherein,
R³ and R⁴ are independently hydrogen, or unsubstituted C₁-C₈ alkyl;
A² is substituted or unsubstituted heterocycloalkyl, substituted or unsubstituted heteroaryl, or substituted or unsubstituted fused ring.

6. The compound of claim 1, wherein
X¹ and X² are O; and
R¹ and R² are independently hydrogen, halogen, substituted or unsubstituted C₁-C₂₀ alkyl, substituted or unsubstituted 2 to 20 membered heteroalkyl, C₃-C₈ substituted or unsubstituted cycloalkyl, 3 to 8 membered substituted or unsubstituted heterocycloalkyl, substituted or unsubstituted aryl, or substituted or unsubstituted heteroaryl.

7. The compound of claim 1, wherein
R¹ and R² are independently hydrogen, halogen, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl.

8. The compound of claim 1, wherein R¹ and R² are hydrogen.

9. The compound of claim 5, wherein
A¹ is substituted or unsubstituted aryl, substituted or unsubstituted heteroaryl, or substituted or unsubstituted fused ring; and
A² is substituted or unsubstituted fused ring.

10. The compound of claim 5, wherein
A¹ is unsubstituted aryl, unsubstituted heteroaryl, or unsubstituted fused ring; and
A² is unsubstituted fused ring.

11. The compound of claim 5, wherein
A¹ is unsubstituted phenyl or unsubstituted naphthalenyl; and
A² is unsubstituted fused ring aryl.

12. The compound of claim 5, wherein
A¹ is unsubstituted phenyl or unsubstituted naphthalenyl; and
A² is unsubstituted purinyl.

13. The compound of claim 5, wherein R³ and R⁴ are hydrogen.

14. The compound of claim 1, wherein
L² is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene; and
L¹ is a bond, —C(O)-L³-, —O—, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene, wherein
L³ is a bond, —O-L⁴-, or —N(R⁵)-L⁴-, wherein
R⁵ is hydrogen, substituted or unsubstituted C₁-C₂₀ alkyl, substituted or unsubstituted 2 to 20 membered heteroalkyl, C₃-C₈ substituted or unsubstituted cycloalkyl, 3 to 8 membered substituted or unsubstituted heterocycloalkyl, substituted or unsubstituted aryl, or substituted or unsubstituted heteroaryl, and
L⁴ is a bond, substituted or unsubstituted C₁-C₂₀ alkylene, substituted or unsubstituted 2 to 20 membered heteroalkylene, substituted or unsubstituted C₃-C₈ cycloalkylene, substituted or unsubstituted 3 to 8 membered heterocycloalkylene, substituted or unsubstituted arylene, or substituted or unsubstituted heteroarylene.

15. The compound of claim 1, wherein
L² is substituted or unsubstituted C₃-C₈ cycloalkylene, substituted or unsubstituted 3 to 8 membered heterocycloalkylene, substituted or unsubstituted arylene, or substituted or unsubstituted heteroarylene; and
L¹ is —C(O)-L³-, —O—, substituted or unsubstituted C₁-C₂₀ alkylene, substituted or unsubstituted 2 to 20 membered heteroalkylene, wherein
L³ is a bond, —O-L⁴-, or —N(R⁵)-L⁴-, wherein
R⁵ is hydrogen, substituted or unsubstituted C₁-C₂₀ alkyl, or substituted or unsubstituted 2 to 20 membered heteroalkyl, and
L⁴ is a bond, substituted or unsubstituted C₁-C₂₀ alkylene, or substituted or unsubstituted 2 to 20 membered heteroalkylene.

16. The compound of claim 1, wherein
L² is unsubstituted C₅-C₈ cycloalkylene; unsubstituted 5 to 8 membered heterocycloalkylene; unsubstituted arylene; unsubstituted heteroarylene;
C₅-C₈ cycloalkylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene;
5 to 8 membered heterocycloalkylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene;
arylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene; or
heteroarylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene.

17. The compound of claim 1, wherein
L² is unsubstituted C₅-C₈ cycloalkylene; unsubstituted 5 to 8 membered heterocycloalkylene;
C₅-C₈ cycloalkylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene; or
5 to 8 membered heterocycloalkylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene.

18. The compound of claim 1, wherein
L² is unsubstituted 5 to 8 membered heterocycloalkylene; or
C₅-C₈ cycloalkylene substituted with an oxo, —OH, —NH₂, —CN, halogen, unsubstituted C₁-C₁₀ alkyl, unsubstituted 2 to 10 membered heteroalkyl, unsubstituted C₅-C₈ cycloalkylene, unsubstituted 5 to 8 membered heterocycloalkylene, unsubstituted arylene, or unsubstituted heteroarylene.

19. The compound of claim 1, wherein L² is C₅-C₈ cycloalkylene substituted with an oxo, —OH, —NH₂, —CN, or halogen.

20. The compound of claim 1, wherein L² is a ribose ring or deoxyribose ring.

21. The compound of claim 1, wherein
L¹ is a bond, —C(O)-L³-, —O—, unsubstituted C₁-C₂₀ alkylene, unsubstituted 2 to 20 membered heteroalkylene, unsubstituted C₃-C₈ cycloalkylene, unsubstituted 3 to 8 membered heterocycloalkylene, unsubstituted arylene, or unsubstituted heteroarylene, wherein
L³ is a bond, —O-L⁴-, or —N(R⁵)-L⁴-, wherein
R⁵ is hydrogen, unsubstituted C₁-C₂₀ alkyl, unsubstituted 2 to 20 membered heteroalkyl, unsubstituted C₃-C₈ cycloalkyl, unsubstituted 3 to 8 membered heterocycloalkyl, unsubstituted aryl, or unsubstituted heteroaryl, and
L⁴ is a bond, unsubstituted C₁-C₂₀ alkylene, unsubstituted 2 to 20 membered heteroalkylene, unsubstituted C₃-C₈ cycloalkylene, unsubstituted 3 to 8 membered heterocycloalkylene, unsubstituted arylene, or unsubstituted heteroarylene.

22. The compound of claim 1, wherein
L¹ is —C(O)-L³-, unsubstituted C₁-C₂₀ alkylene, or unsubstituted heteroalkylene, wherein
L³ is a bond, —O-L⁴-, or —N(R⁵)-L⁴-, wherein
R⁵ is hydrogen, unsubstituted C₁-C₂₀ alkyl, or unsubstituted heteroalkyl, and
L⁴ is a bond, unsubstituted C₁-C₂₀ alkylene, or unsubstituted 2 to 20 membered heteroalkylene.

23. The compound of claim 1, wherein
L¹ is a bond, —C(O)-L³-, unsubstituted C₁-C₂₀ alkylene, or unsubstituted 2 to 20 membered heteroalkylene, wherein
L³ is a bond, —O-L⁴-, or —N(R⁵)-L⁴-, wherein
R⁵ is hydrogen, or unsubstituted 2 to 20 membered heteroalkylene, and
L⁴ is unsubstituted C₁-C₁₀ alkylene.

24. The compound of claim 1, wherein
L¹ is a bond, —C(O)-L³-, unsubstituted C₁-C₁₀ alkylene, or unsubstituted 2 to 10 membered heteroalkylene, wherein
L³ is a bond, —O-L⁴-, or —N(R⁵)-L⁴-, wherein
R⁵ is hydrogen, and
L⁴ is unsubstituted C₁-C₄ alkylene.

25. The compound of claim 1, wherein L¹ is —C(O)—O—(CH₂)ₙ— or, —C(O)—NH—(CH₂)ₙ— or —(CH₂)ₙ—O—(CH₂)ₙ—, wherein n is an integer from 0 to 10.

26. The compound of claim 1, having the formula 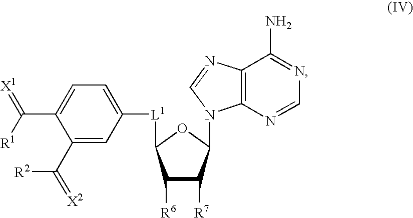
wherein
R⁶ and R⁷ are, independently, hydrogen, halogen, —OH, or —OR⁸, wherein
R⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

27. The compound of claim 26, wherein
R⁸ is unsubstituted C₁-C₅ alkyl, or unsubstituted 2 to 5 membered heteroalkyl;
X¹ and X² are oxygen;
R¹ and R² are hydrogen; and
L¹ is —C(O)—O—CH₂— or, —C(O)—NH—CH₂— or —CH₂—O—CH₂—.

28. The compound of claim 27, wherein R⁶ is —OH, and R⁷ is —OH or hydrogen.

29. The compound of claim 1, having the formula 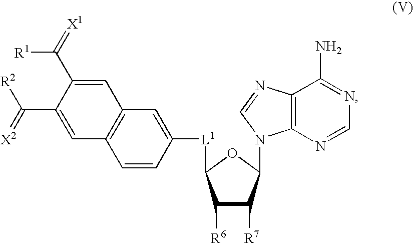
wherein
R⁶ and R⁷ are, independently, hydrogen, halogen, —OH, or —OR⁸, wherein
R⁸ is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

30. The compound of claim 29, wherein
R⁸ is unsubstituted C₁-C₅ alkyl, or unsubstituted 2 to 5 membered heteroalkyl;
X¹ and X² are oxygen;
R¹ and R² are hydrogen; and
L¹ is —C(O)—O—CH₂— or, —C(O)—NH—CH₂— or —CH₂—O—CH₂—.

31. The compound of claim 30, wherein R⁶ is —OH, and R⁷ is —OH or hydrogen.

32. The compound of claim 1 having the formula: 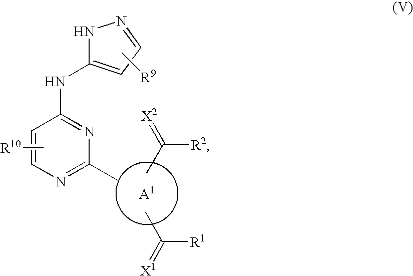
wherein
R⁹ and R¹⁰ are independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

33. A method of detecting binding between an interactor and a kinase comprising the steps of:
(a) contacting a kinase with a mechanism-based crosslinker and an interactor;
(b) allowing the mechanism-based crosslinker to form a covalent bond with the interactor and allowing the mechanism-based crosslinker to specifically form a covalent bond with a catalytic amino acid side chain of the kinase thereby forming a crosslinked kinase-interactor pair; and
(c) detecting the presence of the crosslinked kinase-interactor pair thereby detecting binding between the interactor and the kinase.

34. The method of claim 33, wherein the covalent bond with the catalytic amino acid is formed only in the presence of the interactor.

35. The method of claim 33, wherein the interactor binds to the peptide binding groove of the kinase.

36. The method of claim 33, wherein the interactor comprises a thiol moiety that forms the covalent bond with the mechanism-based crosslinker.

37. The method of claim 33, wherein the catalytic amino acid side chain of the kinase is a lysine amino acid side chain of the kinase.

38. The method of claim 36, wherein the mechanism-based crosslinker comprises a first crosslinker reactive group and a second crosslinker reactive group.

39. The method of claim 38, wherein the first crosslinker reactive group and the second crosslinker reactive group are an aldehyde.

40. The method of claim 33, wherein the mechanism-based crosslinker binds to the ATP-binding pocket of the kinase.

41. The method of claim 33, wherein the mechanism-based crosslinker comprises an ATP-binding moiety.

42. The method of claim 33, wherein the crosslinked kinase-interactor pair is luminescent.

43. A method of detecting an active kinase in a sample, the method comprising the steps of:
(a) contacting an array of immobilized interactors with a mechanism-based crosslinker and a sample comprising an active kinase;
(b) allowing the mechanism-based crosslinker to form a covalent bond with the interactor and specifically form a covalent bond with a catalytic amino acid side chain of the active kinase, thereby forming an immobilized crosslinked kinase-interactor pair; and
(c) detecting the presence of the immobilized crosslinked kinase-interactor pair thereby detecting said active kinase.

44. The method of claim 43, wherein the mechanism-based crosslinker is the compound of claim 1.
